(19) United States
(12) Patent Application Publication
(10) Pub. No.: US 2025/0020635 A1
(43) Pub. Date: Jan. 16, 2025

(54) ASSAY METHODS AND USES THEREOF FOR SCREENING ALZHEIMER'S DISEASE TREATMENT COMPOUNDS

(71) Applicant: The Trustees Of Indiana University, Bloomington, IN (US)

(72) Inventors: Shaoyou Chu, Carmel, IN (US); Emily Mason, Carmel, IN (US)

(21) Appl. No.: 18/769,365

(22) Filed: Jul. 10, 2024

Related U.S. Application Data
(60) Provisional application No. 63/513,049, filed on Jul. 11, 2023.

(51) Int. Cl. G01N 33/50; G01N 1/34; G01N 21/64; G01N 33/58

(52) U.S. Cl. CPC G01N 33/5047; G01N 1/34; G01N 21/6428; G01N 33/502; G01N 33/582; G01N 2021/6439

(57) ABSTRACT

The present disclosure provides methods, assays, kits, and uses thereof for screening candidate therapeutics for their tendency to induce phagocytosis in a cell. In one aspect, the present disclosure provides a method of assaying a candidate compound for its tendency to induce phagocytosis in a cell, the method comprising: contacting the cells with the candidate compound; contacting the cells with a target ligand bound to a fluorescent phagocytosis vesicle reporter having a fluorophore emission wavelength; nuclear staining the cells with a DNA-specific fluorescent stain; and measuring the fluorescence amplitude of the fluorescent phagocytosis vesicle reporter. In another aspect, the present disclosure provides a cell-health assay for testing compounds for their potential for treating neurodegenerative disorders.

### CROSS-REFERENCE TO RELATED APPLICATIONS

The present application claims priority to U.S. Patent Application No. 63/513,049, filed on Jul. 11, 2023, which is incorporated by reference herein in its entirety.

### STATEMENT OF FEDERALLY SPONSORED RESEARCH OR DEVELOPMENT

This invention was made with government support under AG065181 awarded by National Institutes of Health. The Government has certain rights in the invention.

### BACKGROUND

Neurodegenerative disorders represent a significant unmet medical need, affecting millions of patients worldwide.

The present disclosure addresses this unmet need. The disclosure relates to new methods of screening and predicting the therapeutic potential of compounds for their usefulness in treating neurodegenerative disorders such as Alzheimer's disease (AD).

### SUMMARY

The present disclosure relates methods of the identification of small molecules to activate 1-phosphatidylinositol 4,5-bisphosphate phosphodiesterase gamma-2 (PLCγ2) enzyme as a therapeutic strategy to reduce rate of cognitive decline in Alzheimer's patients. This disclosure shows that general PLC activator m-3M3FBS (2,4,6-trimethyl-N-(meta-3-trifluoromethyl-phenyl)-benzenesulfonamide) and certain analogs are not statistically efficacious or selective towards PLCγ2 activation; and that high throughput screening of a large number of compounds (e.g., ˜50,000 compounds) for PLCγ2 affinity and activation leads to discovery of compounds that activate PLCγ2, leading to the identification of compounds that are statistically efficacious and selective towards PLCγ2 activation.

In an aspect, the present disclosure provides a method of assaying a candidate compound for its tendency to induce phagocytosis in a cell, the method comprising: (a) contacting the cells with the candidate compound; (b) contacting the cells with a target ligand bound to a fluorescent phagocytosis vesicle reporter having a fluorophore emission wavelength; (c) nuclear staining the cells with a DNA-specific fluorescent stain; and (d) measuring the fluorescence amplitude of the fluorescent phagocytosis vesicle reporter at a measurement wavelength corresponding to the fluorophore emission wavelength.

The cells may be any suitable cell type. In any embodiment, the cells may be microglia. In any embodiment, the target ligand may be myelin, cell membrane debris, synaptosomes, zymosan, bacteria, protein aggregates (Aβ, Tau), microbeads, and any combination thereof. In any embodiment, the cell membrane debris may be from brain tissue. In any embodiment, the cell membrane debris may be from mouse brain tissue.

In any embodiment, the fluorescent phagocytosis vesicle reporter may be a fluorogenic, cell-permeant pH indicator that is non-fluorescent at a neutral pH and increases in intensity with an increase in acidity, and having a peak excitation and emission wavelength at about 560 nm and about 585 nm, respectively.

In any embodiment, the DNA-specific fluorescent stain may be Hoechst 33342. In any embodiment, the DNA-specific fluorescent stain may comprise a cell-membrane permeable DNA-specific fluorophore.

In any embodiment, the candidate compound may be an analog of 2,4,6-trimethyl-N-(m-tolyl)benzenesulfonamide. In any embodiment, the candidate compound may be an analog of (2,4,6-trimethyl-N-(meta-3-trifluoromethyl-phenyl)-benzenesulfonamide).

In another aspect, the present disclosure provides a cell-health assay for testing compounds for their potential for treating neurodegenerative disorders, the assay comprising: (1) plating microglia cells in one or more microplate wells; (2) incubating the microglia cells; (3) contacting the microglia cells with one or more candidate compounds; (4) adding fluorescent-dye-labeled-myelin/membrane debris to the cell plates, wherein the fluorescent-dye-labeled-myelin/membrane debris comprises crude myelin/membrane debris from 6-8-week-old wild type mouse brains isolated via sucrose gradient ultracentrifugation and subsequently labeled with fluorescent dye; (5) staining the microglia cells' nuclei with a DNA-binding fluorescent dye; (6) scanning the microglia cells with a fluorescence-reading instrument; and (7) measuring one or more of (i) mean total fluorescence intensity of phagocytosis vesicles per cell; (ii) cell count per well; and (iii) average nuclear fluorescence intensity.

In any embodiment of the assay, the microglia may be BV2 cells, HMC3 cells, a mixture of BV2 cells and HMC3 cells, or mouse primary microglia.

In any embodiment, the microglia cells may be incubated at 37° C. with 5% CO₂ for 16 to 24 hours.

In any embodiment, the fluorescent dye may be a fluorogenic, cell-permeant dye with an excitation peak at about 560 nm and emission peak at about 585 nm.

In any embodiment, the DNA-binding fluorescent dye may be Hoechst 33342.

In any embodiment, scanning the microglia cells is performed using a high-content analysis (HCA) quantitative cell analysis apparatus having at least a first channel set to scan for an excitation peak of about 386 nm and emission peak of about 460 nm, and a second channel set to scan for an excitation peak of about 560 nm and an emission peak of about 585 nm.

In still another aspect, the present disclosure provides a kit for carrying out a phagocytosis assay for testing a compound for its tendency to modulate and/or regulate phagocytosis in microglia, the kit comprising: one or more well plates; immortalized microglia cells; and a ligand for measuring microglial phagocytosis, wherein the ligand is labeled with a fluorogenic, cell-permeant dye with an excitation peak at about 560 nm and emission peak at about 585 nm, which is essentially non-fluorescent or non-fluorescent at neutral pH and which fluoresces at increasing intensity correlated with increasing acidity. In any embodiment, the ligand may be selected from myelin, cell membrane debris, Aβ protein aggregates, Tau protein aggregates, microbeads, zymosan, bacteria, isolated dead neurons, synaptosomes, or any combination thereof. In any embodiment, the ligand may be myelin/membrane debris isolated from 6-8-week-old wild-type mouse brain. In any embodiment of the kit, the immortalized microglia cells may be BV2 cells, HMC3 cells, or a mixture of BV2 cells and HMC3 cells. In any embodiment, the kit may further comprise a cell-membrane-permeable DNA-binding fluorescent dye. In any embodiment, the dye may be Hoechst 33342. In any embodiment, the kit may further comprise instructions describing how to perform the methods and assays of the present disclosure.

### BRIEF DESCRIPTION OF THE DRAWINGS

The details of one or more embodiments of the invention are set forth in the accompanying drawings and the description below. Other features, objects, and advantages of the invention will be apparent from the description and drawings, and from the claims.

FIG. 1 is a diagram showing various increases and decreases effectuated by the PLCG2-P522R variant versus wild-type.

FIGS. 2A-2D depict the model workflow used in Example 1 of the present disclosure. FIG. 2A is a diagram modeling screening by affinity selection mass-spectrometry (ASMS). FIG. 2B is exemplary CEllular Thermal Shift Assay (CETSA) output data. FIG. 2C is an exemplary candidate molecule and liposome. FIG. 2D is a flow diagram describing the steps of the model workflow.

FIG. 3 is a funnel diagram exemplifying the winnowing of high-throughput screened molecules from ˜50,000 to 2 lead candidates.

FIGS. 4A-4C depict CETSA (FIG. 4A), XY69 (FIG. 4B), and phagocytosis (FIG. 4C) assay results for compound TAD-0411060.

FIGS. 5A-5C depict CETSA (FIG. 5A), XY69 (FIG. 5B), and phagocytosis (FIG. 5C) assay results for compound TAD-0410342.

FIGS. 6A-6B depict plate maps of compound serial dilution and treated cell plate. Compound serial dilutions are performed in 96-well compound plates for treating cells in the 384-wellcell plate. Two 96-well plates will be used for compound dilutions to cover one 384-well cell plates for testing 16 compounds in duplicates. An example plate map of compound dilution is shown in FIG. 6A and a treated cell plate map shown in FIG. 6B.

FIG. 7 is a graph of pHrodo™-myelin/membrane debris concentration dependent phagocytosis results. pHrodo™-myelin/membrane debris concentration-dependent phagocytosis was tested in both HMC3 and BV2 cells through 4-hr exposures. Both cells showed a concentration-dependent phagocytosis signal: 5 μg/ml (>50% of maximal signal) was chosen for the assay.

FIGS. 8A-8D are micrographs showing characterization of pHrodo™-myelin/membrane debris. The pHrodo™-myelin/membrane debris was checked in PBS for fluorescence and on cells for phagocytosis. Overlayed transmitted light and fluorescent images are shown. FIG. 8A: in pH-6.94 PBS with no red fluorescence; FIG. 8B: in pH-4.28 PBS with strong red fluorescence. FIG. 8C: HMC3 cells with red phagocytosis vesicles. FIG. 8D: BV2 cells with red phagocytosis vesicles. Scale bar=100 μm.

FIGS. 9A-9C are exemplary images from a 384-well assay plate. The plate image (FIG. 9A) (a montage of a 384-well assay plate) shows the experimental design; 2 columns of Max control wells with pHrodo™-myelin/membrane debris seeding (FIG. 9B), 2 columns of Min of unseeded wells (FIG. 9C); and 16 duplicate rows for testing compounds in 10-point concentration response. Two zoomed in images are examples from a Max well (FIG. 9B) and a Min well (FIG. 9C) showing with phagocytosis vesicles (red) and nuclei (blue).

FIG. 10 is a series of micrographs showing image analysis of the phagocytosis/cell health assay. example images are from HMC3 and BV2 cells with (Max, 5 g/ml) and without (Min, 0 μg/ml) pHrodo™-myelin/membrane debris seeding. Original images were segmented by 3 steps, 1) identify individual nucleus shown by a blue line on nuclear edge; 2) expand the nuclear edge to cover cell area shown by green lines around each cell; 3) identify and mask phagocytosis vesicles (masking shown in yellow) in cell area. Arrows show the transformation of original images to segmented and masked images used for quantitative measurements. (Red—phagocytosis spots; Blue—nuclei; Green—ROI of single cells; Yellow—masking for phagocytosis spots).

FIG. 11 is a graph showing Cytochalasin D inhibited microglial phagocytosis. HMC3 cell phagocytosis was inhibited by cytochalasin D (IC₅₀=138 nM) in the assay; its cytotoxicity was reflected by the inhibition of cell proliferation (IC₅₀=367 nM) and parallel increase of nuclear intensity (EC₅₀=488 nM).

FIGS. 12A-12B are graphs showing LPS stimulating phagocytosis of BV2 but not HMC3 cells. Cells were treated with serially diluted LPS for 48 hr. (FIG. 12A) HMC3 cells did not respond to LPS treatment, as shown by all three measurements. (FIG. 12B) BV2 cells showed a concentration-dependent stimulation of phagocytosis (EC₅₀=255 ng/ml), an inhibition of cell count (IC₅₀=122 ng/ml), and no changes in nuclear intensity.

FIGS. 13A-13B are graphs showing PI3Kd inhibitor (Idelalisib) regulation of microglial phagocytosis. FIG. 13A: Idelalisib (48 hours treatment) inhibited phagocytosis in HMC3 cells (IC₅₀=526 nM), while not affecting cell count or nuclear intensity. Results indicated that Idelalisib specifically inhibited HMC3 phagocytosis. FIG. 13B: BV2 cells showed a concentration-dependent cell count decrease (IC₅₀=22 μM), nuclear intensity increase (EC₅₀=4.14 μM), and parallel stimulation of phagocytosis (EC₅₀>60 μM, the maximal dose in the assay), indicating that the Idelalisib stimulated phagocytosis of BV2 cells was cell stress related rather than specific activity.

FIG. 14A-14B are graphs showing Src inhibitor (Saracatinib) regulation of microglial phagocytosis. FIG. 14A. Saracatinib (48 hours treatment) stimulated HMC3 cell phagocytosis (EC₅₀=182 nM), with minimal effects on cell count and nuclear intensity until concentrations >10 μM showing cytotoxicity. Results indicated that Saracatinib specifically stimulated HMC3 phagocytosis. Example images selected from 3 dose points showed cells at normal state (A1, healthy cells), at stimulated phagocytosis state (A2, healthy cells) and cells with nuclear condensation and no phagocytosis (A3, unhealthy cells in apoptosis, marked by arrows) FIG. 14B. BV2 cells showed a dose dependent cell count decrease (IC₅₀=833 nM) and parallel nuclear intensity changes (increase then decrease), as well as increased phagocytosis (EC₅₀=266 nM). Selected images showed healthy normal cells (B1), cells under stress with increased phagocytosis (B2) and dead cells with low nuclear intensity since DNA degradation (B3, marked by arrows). Blue—nuclei; Red—phagocytosis vesicles.

FIGS. 15A-15C are primary (FIG. 15C) and 3-dimensional (FIG. 15B) models of PLCG2 protein and PI(4,5) P2 linkages (FIG. 15A).

FIG. 16 is an exemplary proposed mechanism of PLCγ enzymes transitioning from the autoinhibited to the active state. (Created in BioRender.)

FIG. 17A-17D are a series of representative CETSA dose response curves for PLCγ2-HiBit (blue) and HiBit-only (black) for m-3M3FBS (FIG. 17A), TAD-0058715 (FIG. 17B), TAD-0410993 (FIG. 17C), and TAD-0411056 (FIG. 17D).

FIG. 18A-18C is exemplary affinity selection mass spectrometry (ASMS) data for PLCγ1 (red), PLCγ2 (blue), and background (black), for each of TAD-0000040 (FIG. 18A), TAD-0000043 (FIG. 18B), and ATP (FIG. 18C).

FIG. 19 is a graph showing fluorescence intensity in a time-dependent response liposomal response assay for compound TAD-0058717 in each of 112.5 μM, 40.0 μM, 12.5 μM, 5.00 μM, 1.00 μM, 0.325 μM, 0.100 μM, 0.025 μM, and control (0 μM).

FIGS. 20A-20B are graphs showing representative examples of liposomal biochemical assay dose response curves for: 1 (TAD-0000040), 10 (TAD-0411016), 6 (TAD-0058717), 3 (TAD-0058693), 9 (TAD-0410993) (FIG. 20A); and for (TAD-0058725), 5 (TAD-0058715), 2 (TAD-0000043), 4 (TAD-0058698), 8 (TAD-0410954) (FIG. 20B).

FIGS. 21A-21D are graphs showing phagocytosis in model cell line HMC3 for each of compound TAD-0000040 (FIG. 21A), TAD-0000043 (FIG. 21B), TAD-0058715 (FIG. 21C), TAD-0411056 (FIG. 21D), where blue is cell count, grey is nuclear intensity, and red is phagocytosis.

FIGS. 22A-22D are graphs showing are graphs showing phagocytosis in model cell line BV2 for each of compound TAD-0000040 (FIG. 22A), TAD-0000043 (FIG. 22B), TAD-0058715 (FIG. 22C), TAD-0411056 (FIG. 22D), where blue is cell count, grey is nuclear intensity, and red is phagocytosis.

FIGS. 23A-23D are graphs showing phagocytosis in primary mouse microglia cells for each of compound TAD-0058698 (FIG. 23A), TAD-0058717 (FIG. 23B), TAD-0058725 (FIG. 23C), TAD-0411056 (FIG. 23D), where blue is cell count, grey is nuclear intensity, and red is phagocytosis.

FIG. 24 is a model of PLCG2 pathway.

FIG. 25 is a model of PLCG2's conformational change.

FIG. 26 depicts m-3mFCS analogs generated by TREAT-AD for assessment as potential PLC activators.

FIGS. 27A-27B are graphs showing the ability for candidate compound TAD-0058725 to activate BV2 (FIG. 27A) cells and mouse microglia (FIG. 27B).

FIG. 28 depicts chemical structures of structures of m-3M3FBS, o-3M3FBS, and structural analogs.

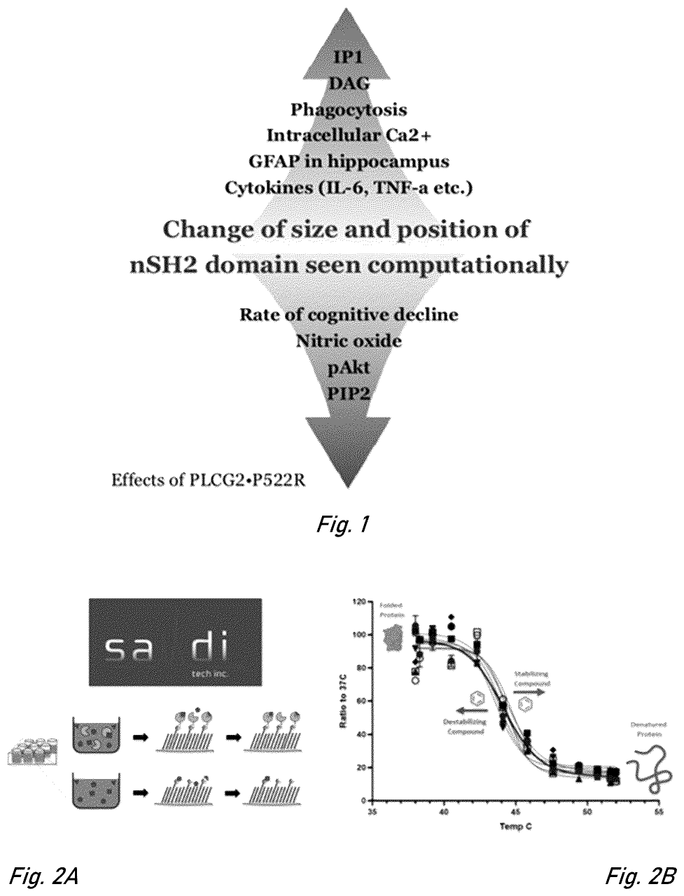

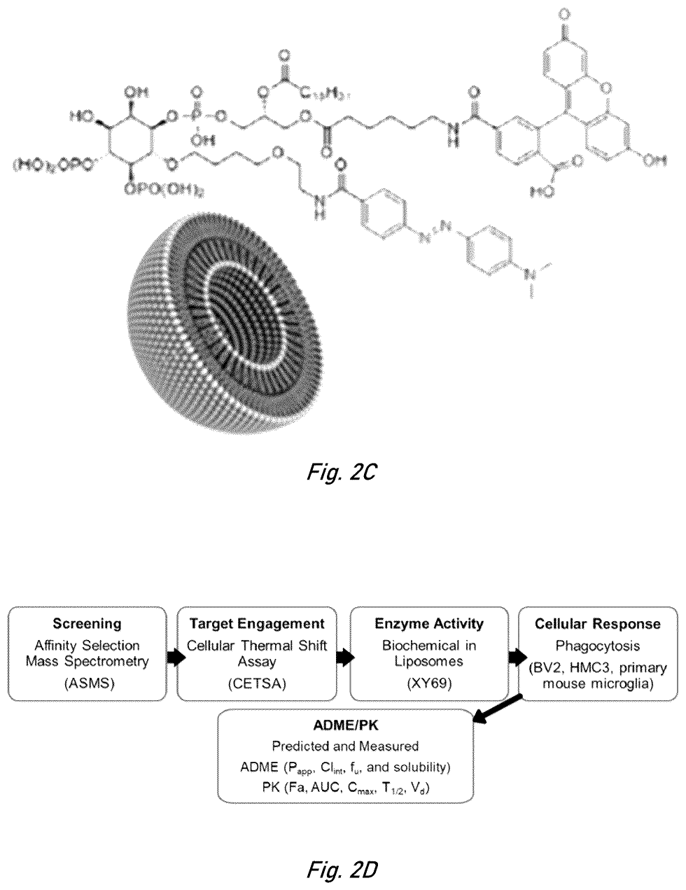

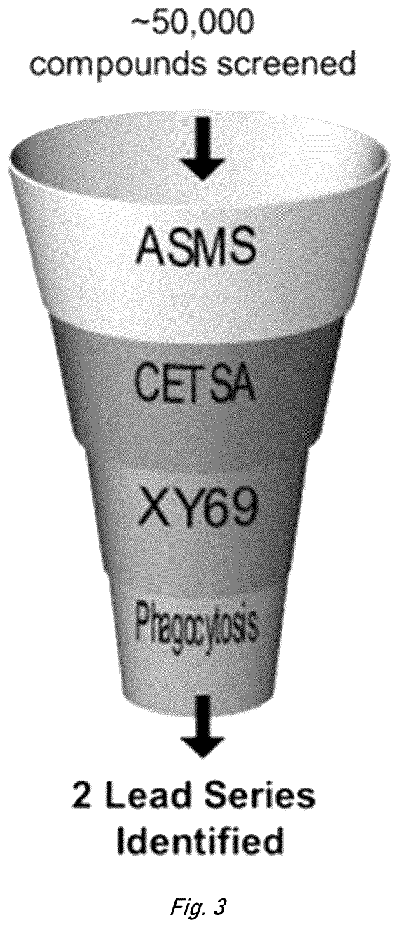

### DETAILED DESCRIPTION

To develop effective pharmacological treatments for Alzheimer's disease, numerous genome-wide association studies and genetic analyses have compared brain tissues from normal aged patients to patients diagnosed with late-onset Alzheimer's disease, identifying protective and risk-associated genes. 1-phosphatidylinositol 4,5-bisphosphate phosphodiesterase gamma-2 (PLCγ2) is a member of the phospholipase C (PLC) enzyme family that cleaves phosphatidylinositol 4,5-bisphosphate (PI(4,5) P2) into its two effectors 1D-myo-inositol 1,4,5-trisphosphate (IP3) and diacylglycerol (DAG), propagating various downstream signals. It is largely expressed in immune cells and microglia, playing a role in many cellular responses including phagocytosis and related inflammatory and immune response processes. A rare variant of PLCγ2, known as PLCG2P522R, has been shown to have a protective effect, as Alzheimer's patients with the variant display a slower rate of cognitive decline compared to other patients. This variant codes for a functionally hypermorphic version of the enzyme. Therefore, the use of pharmacological treatments to increase activity of wild-type PLCγ2 can act as a therapeutic treatment to attenuate the rate of cognitive decline in Alzheimer's patients.

Currently, only two molecules have been described as PLC activators: U73122 (1-(6-((17β-3-methoxyestra-1,3,5 (10)-trien-17-yl)amino)hexyl)-1H-pyrrole-2,5-dione) and m-3M3FBS (2,4,6-trimethyl-N-(meta-3-trifluoromethyl-phenyl)-benzenesulfonamide). While m-3M3FBS is a known activator of the general PLC family, presently no small molecules have been identified as being selective towards activation of PLCγ2. The aim of the present disclosure is to determine if structural analogs of m-3M3FBS can be identified as potent and selective activators of PLCγ2.

A high throughput screening paradigm was created to analyze the ability of these compounds to target PLCγ2 and to analyze their biochemical activity. Cellular thermal shift assays (CETSA) and affinity selection mass spectrometry (ASMS) were first used to assess the affinity of these compounds for the target. The majority of analogs displayed responses when PLCγ2 was not present, suggesting promiscuity of these compounds; in other words, a lack of specificity towards this target. Further studies were conducted to measure the activity of PLCγ2 in the presence of liposomes (reminiscent of the cell membrane where PLCγ2 is endogenously active); measure rate of phagocytosis (since PLCγ2 mediates this process); and general cell health after exposure to analogs. In terms of PLCγ2 activity, the highest increase in response was seen in m-3M3FBS itself, with little or no activity recorded in other analogs. Ideally, if PLCγ2 is activated, an increase in phagocytosis would be detected, but the majority of the analogs displayed no significant increase in activity. The few compounds that did lead to an increase in phagocytosis, also caused a significant amount of cell loss indicating cytotoxicity.

Overall, the analogs of m-3M3FBS failed to show significant selectivity and potency towards PLCγ2. Throughout this process around 50,000 compounds were screened, which did lead to the discovery of two lead scaffolds, serving as a starting point for the development of effective and selective PLCγ2 activators.

In an aspect, the present disclosure provides a method of assaying a candidate compound for its tendency to induce phagocytosis in a cell, the method comprising: (a) contacting the cells with the candidate compound; (b) contacting the cells with a target ligand bound to a fluorescent phagocytosis vesicle reporter having a fluorophore emission wavelength; (c) nuclear staining the cells with a DNA-specific fluorescent stain; and (d) measuring the fluorescence amplitude of the fluorescent phagocytosis vesicle reporter at a measurement wavelength corresponding to the fluorophore emission wavelength.

The cells may be any suitable cell type. In any embodiment, the cells may be microglia. In any embodiment, the target ligand may be myelin, cell membrane debris, synaptosomes, zymosan, bacteria, protein aggregates (Aβ, Tau), microbeads, and any combination thereof. In any embodiment, the cell membrane debris may be from brain tissue. In any embodiment, the cell membrane debris may be from mouse brain tissue.

In any embodiment, the fluorescent phagocytosis vesicle reporter may be a fluorogenic, cell-permeant pH indicator that is essentially non-fluorescent or non-fluorescent at a neutral pH and increases in intensity with an increase in acidity, and having an excitation and emission wavelength at about 560 nm and 585 nm, respectively. In any embodiment, the fluorescent phagocytosis vesicle reporter may be a fluorogenic, cell-permeant pH indicator that is non-fluorescent at a neutral pH and increases in intensity with an increase in acidity, having excitation and emission wavelength at 560 nm and 585 nm, respectively.

In any embodiment, the DNA-specific fluorescent stain may be Hoechst 33342. In any embodiment, the DNA-specific stain may comprise 4′,6-diamidino-2-phenylindole (DAPI). In any embodiment, the DNA-specific stain may comprise any suitable cell-membrane-permeable DNA binding fluorophores.

In any embodiment, the candidate compound may be an analog of 2,4,6-trimethyl-N-(m-tolyl)benzenesulfonamide. In any embodiment, the candidate compound may be an analog of 2,4,6-trimethyl-N-(meta-3-trifluoromethyl-phenyl)-benzenesulfonamide.

In another aspect, the present disclosure provides a cell-health assay for testing compounds for their potential for treating neurodegenerative disorders, the assay comprising: (1) plating microglia cells in one or more microplate wells; (2) incubating the microglia cells; (3) contacting the microglia cells with one or more candidate compounds; (4) adding fluorescent-dye-labeled-myelin/membrane debris to the cell plates, wherein the fluorescent-dye-labeled-myelin/membrane debris comprises crude myelin/membrane debris from 6-8-week-old wild type mouse brains isolated via sucrose gradient ultracentrifugation and subsequently labeled with fluorescent dye; (5) staining the microglia cells' nuclei with a DNA-binding fluorescent dye; (6) scanning the microglia cells with a fluorescence-reading instrument; and (7) measuring one or more of (i) mean total fluorescence intensity of phagocytosis vesicles per cell; (ii) cell count per well; and (iii) average nuclear fluorescence intensity. In any embodiment, step (7) may comprise measuring (i) mean total fluorescence intensity of phagocytosis vesicles per cell; (ii) cell count per well; and (iii) average nuclear fluorescence intensity.

In any embodiment of the assay, the microglia may be BV2 cells, HMC3 cells, a mixture of BV2 cells and HMC3 cells, or primary mouse microglia. In any embodiment of the assay, the microglia may be freshly harvested mammalian microglia. In any embodiment of the assay, the microglia may be freshly harvested mouse microglia.

In any embodiment, the microglia cells may be incubated at 37° C. with 5% CO₂ for 16 to 24 hours.

In any embodiment, the fluorescent dye may be a fluorogenic, cell-permeant dye with an excitation peak at about 560 nm and emission peak at about 585 nm. In any embodiment, the fluorescent dye may be a fluorogenic, cell-permeant dye with an excitation peak at 560 nm and emission peak at 585 nm.

In any embodiment, the DNA-binding fluorescent dye may be DAPI or Hoechst 33342. In any embodiment, the DNA-binding fluorescent dye may be Hoechst 33342.

In any embodiment, scanning the microglia cells is performed using a high-content analysis (HCA) quantitative cell analysis apparatus having at least a first channel set to scan for an excitation peak of about 386 nm and emission peak of about 460 nm, and a second channel set to scan for an excitation peak of about 560 nm and an emission peak of about 585 nm. For example, the scanning step may be performed using an ArrayScan™ XTI High Content Analysis (ThermoFisher) system.

In still another aspect, the present disclosure provides a kit for carrying out a phagocytosis assay for testing a compound for its tendency to modulate and/or regulate phagocytosis in microglia, the kit comprising: one or more well plates; immortalized microglia cells; and a ligand for measuring microglial phagocytosis, wherein the ligand is labeled with a fluorogenic, cell-permeant dye with an excitation peak at about 560 nm and emission peak at about 585 nm, which is essentially non-fluorescent or non-fluorescent at neutral pH and which fluoresces at increasing intensity correlated with increasing acidity. In any embodiment, the ligand may be selected from myelin, cell membrane debris, Aβ protein aggregates, Tau protein aggregates, microbeads, zymosan, bacteria, isolated dead neurons, synaptosomes, or any combination thereof. In any embodiment, the ligand may be myelin/membrane debris isolated from 6-8-week-old wild-type mouse brain. In any embodiment, the well plates may be 24-well plates, 48-well plates, 96-well plates, 192-well plates, or 384-well plates. In any embodiment of the kit, the immortalized microglia cells may be BV2 cells, HMC3 cells, or a mixture of BV2 cells and HMC3 cells. In any embodiment, the kit may further comprise a cell-membrane-permeable DNA-binding fluorescent dye. In any embodiment, the dye may be Hoechst 33342. In any embodiment, the kit may further comprise instructions describing how to perform the methods and assays of the present disclosure.

### EXAMPLES

### Example 1

### Background

1-Phosphatidylinositol-4,5-bisphosphate phosphodiesterase gamma-2 (PLCγ2) is a membrane-associated enzyme and key signal transduction mediator downstream of immune cell receptors reported to be associated with late-onset Alzheimer's disease (LOAD; International Genomics of Alzheimer's Project (IGAP) GWAS data. PLCγ2 mediates TREM2 signaling in microglia; produces second messengers by hydrolyzing phosphatidylinositol (3,4)-bisphosphate (PI(4,5) P2) to diacylglycerol (DAG) and inositol 1,4,5-trisphosphate (IP3), releasing intracellular Ca²⁺ and activating microglia; predominantly exists in an autoinhibited state. PLCγ2 undergoes a conformational change to its activated state through a series of protein interactions, phosphorylation, and breakage of key interdomain interactions.

### Impact of the PLCG2-P522R Variant

PLCG2-P522R is rare genetic variant that is protective against LOAD and has been characterized as a hypermorph. Other variants, such as M28L, confer AD risk. Gain-of-function mutations R665W, S707Y, and M1141L are linked to PLAID and cancer.

### Aim/Hypothesis

This example aimed to identify a small molecule capable of mimicking the P522R-related effects of mild activation and changes in phenotype. This approach is hypothesized to provide protection against AD without the negative effect of the hyperactivating mutations.

### Preliminary Screening

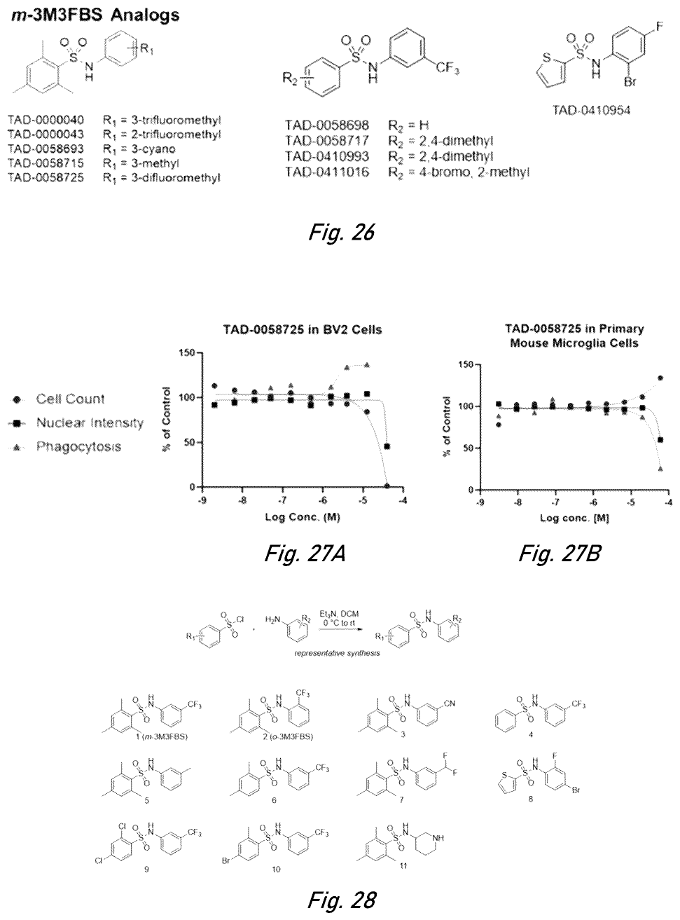

The present example tested the reported PLC activator m-3M3FBS and several analogs we prepared (FIG. 26); results were observed in our assay flow scheme assessing the binding of the ligands to the target, their enzymatic activity (see FIGS. 20A-20B), and ability to elicit microglial activation (FIGS. 27A-27B).

### High-Throughput Screening

The results obtained with the initial analogs necessitated the screening of additional targets; thus, ˜50,000 new ligands were screened with our assay flow scheme for their target affinity and ability to mimic the effects of the PLCG2 P522R variant.

Ligands are screened for target affinity (ASMS) and target binding (CETSA). CETSA assesses the active binding of a ligand to the target protein in a cellular environment. Those exhibiting the desired target interaction are then screened for enzymatic activity in lysosomes and their ability to elicit the desired cellular responses. ADME/PK is used to determine how the ligand is processed. Two lead series were identified as potential mimics of the PLCG2-P522R effect and further evaluated in vitro.

CETSA results are shown in Table 1 below:

TABLE 1
Cellular Thermal Shift Assay (CETSA)

| | | PLCy2-HiBit | HiBit only |
|---|---|---|---|
| Molecule ID | AC50 (μM) | % @100 | % @100 μM |
| TAD-0000040 | NC | 105 | N.T. |
| TAD-0000043 | NC | 102 | N.T. |
| TAD-0058693 | NC | 96 | N.T. |
| TAD-0058698 | NC | 65 | 82 |
| TAD-0058715 | >100 | 54 | 96 |
| TAD-0058717 | 99 | 51 | 74 |
| TAD-0058725 | NC | 78 | 104 |
| TAD-0410954 | >100 | 66 | 44 |
| TAD-0410993 | 71 | 32 | 38 |
| TAD-0411016 | 57 | 17 | 40 |

### Summary

Genetic evidence associates the phospholipase PLCG2 with AD risk, and the hypermorphic rare variant P522R is protective. A reported PLC activator m-3M3FBS and analogs we prepared were tested; results were observed in our assay flow scheme. Screening ˜50,000 compounds for PLCG2 affinity and activation resulted in the discovery of two lead scaffolds.

The assay paradigm developed by TREAT-AD has been established for the optimization of the two PLCG2+P522R series and discovery of new starting points.

### Example 2

### Introduction

Microglia, the resident immune cells of the brain parenchyma, play critical functions to maintain homeostasis in the brain tissue environment, ensuring normal neuronal activities (Kettenmann et al., 2011; Nimmerjahn et al., 2005; Salter & Stevens, 2017; Wake et al., 2013). Cleaning debris of dead cells and extracellular abnormal protein aggregates through phagocytosis is the major homeostasis function of microglia (Edler et al., 2021; Ennerfelt et al., 2022; Franco-Bocanegra et al., 2021; Mundt et al., 2022; Nizami et al., 2019; Witting et al., 2000). Decline of phagocytosis activity has been observed in Alzheimer's disease (AD) brains (Lewcock et al., 2020; Mundt et al., 2022), especially in plaque associated microglia (Gyoneva et al., 2016; Plescher et al., 2018). Accumulation of Aβ plaques and Tau tangles in AD brains suggest that microglia lack the capacity to maintain homeostasis in diseased brains, and that this deficiency contributes to the neurodegenerative pathology leading to dementia. We hypothesize that targeting microglia with therapeutic small molecule agents to stimulate phagocytosis may be a potential therapeutic strategy for AD. Selected targets include main regulators of TREM2 signaling pathway, such as INPP5D and PLCγ2, which have been identified as risk factors related to AD pathogenesis (Andreone et al., 2020; Ennerfelt et al., 2022; Lewcock et al., 2020; Nugent et al., 2020). Since TREM2 regulates microglial phagocytosis activities, quantitative measurement of phagocytosis can be a phenotypic assay to test cellular efficacy for drug discovery projects targeting the pathway. Safety of potential drug molecules is another important factor to be monitored during drug discovery studies. Learning from various reported assays measuring phagocytosis via cellular imaging (Andreone et al., 2020; Phanse et al., 2012; Vance et al., 2016; Witting et al., 2000; Yeo et al., 2013) or flow cytometry (Harvath & Terle, 1999; Meena et al., 2021; Phanse et al., 2012), we developed and established a cellular assay that can measure both the efficacy by quantifying microglial phagocytosis, and simultaneously measure cell health, while maintaining the capacity and reproducibility required for guiding the drug discovery process. This microglial phagocytosis/cell health high-content assay is presented here. The simultaneous measurements of phagocytosis and cell health enable the identification of regulation of microglial phagocytosis in the absence of cell stress/toxicity caused by compounds, a distinguishing feature of the assay. The combination of cell counts and nuclear intensity for cell health is also an effective way to measure cell stress and compound cytotoxicity, and this strategy of simultaneous profiling measurements may have broad applications for drug discovery studies.

### Basic Protocol: Microglial Phagocytosis/Cell Health High Content Assay Protocol

The assay introduced here is a simplified cellular imaging assay without any fixation and washing steps, while producing multiparameter measurements with high capacity using a 384-well plate format. The assay procedures include plating cells on day 1, incubating cell plates overnight before treating cells on day 2, adding pHrodo™-myelin/membrane debris to cell plates on day 3, and 20 hr later, staining cell nuclei with Hoechst-33342 for 30 min before performing high-content imaging on day 4. Three selected parameters are measured from cells: 1) mean total fluorescence intensity per cell of pHrodo™-myelin/membrane debris in phagocytosis vesicles to quantify phagocytosis; 2) cell counts per well to quantify treatment effects on proliferation and cell death; and 3) average nuclear intensity to quantify treatment-induced nuclear changes such as in apoptosis. The assay has been used on HMC3 cells (an immortalized human microglial cell line), BV2 cells (an immortalized mouse microglial cell line), and primary microglia isolated from mouse brains. For initial assay attempts, we recommend using HMC3 cells available from the ATCC.

### Materials

HMC3 microglial cell line (ATCC, cat. no. CRL-3304) (Janabi et al., 1995), or BV2 immortalized mouse microglial cell line (kindly gifted by Dr. Michelle Block. Indiana University School of Medicine) (Blasi et al., 1990; Taetzsch et al., 2015), or primary microglia isolated from C57BL/6J mouse brain (Saura et al., 2003).

### Maintenance Medium (See Recipe Infra);

### Assay Medium (See Recipe Infra);

TrypLE Express (ThermoFisher Scientific, cat. no. 12605-028); Dulbecco's phosphate-buffered saline (DPBS, 1×) no calcium, no magnesium (ThermoFisher Scientific, cat. no. 14190-144) 0.4% trypan blue solution (Gibco, cat. no. 15250061); Sterile water: filter water using MilliQ Millipak 0.22-μm filter (Millipore Sigma, cat. no. MPGP002A1), then autoclave; Serum-free medium: 500 ml DMEM, high glucose, GlutaMAX supplement (ThermoFisher Scientific, cat. no. 10566-016); 70% ethanol; Lipopolysaccharides (LPS) (Sigma-Aldrich cat. no. L2143); Cytochalasin D (ENZO, cat. no. MBL-T109-0001); Idelalisib (Medchemexpress, cat. no. HY-13026); Saracatinib (Medchemexpress, cat. no. HY-10234); DMSO, ≥99.9% (Sigma-Aldrich, cat. no. 276855-100 mL); Hoechst 33342 (Invitrogen, cat. no. H3570); pHrodo™-myelin/membrane debris (1 mg/ml, 55 μL-aliquots) (see Support Protocol infra for preparation); CellXpert C170i cell culture incubator (Eppendorf, cat. no. 2231000868); Biosafety cabinet: 1300 series class II, type A2 biological safety cabinet (ThermoFisher Scientific, cat. no. 1323TS, model 1371); T-75 flasks (uncoated) (Fisher Scientific, cat. no. FB012937); 2-ml microcentrifuge tubes (Fisher Scientific, cat. no. 05-408-138); 10 ml serological pipettes (Fisher Scientific, cat. no. 13-678-11E); Countess II automated cell counter (ThermoFisher Scientific, cat. no. AMQAX 1000); Countess cell counting chamber slides (ThermoFisher Scientific, cat. no. C10283); 50-ml conical tubes (Thermo Scientific, cat. no. 339652); 384-well clear-bottom cell culture plates (Corning, cat. no. 353962); Thermo Scientific Multidrop 384 (cat. no. 5840150 or 5840157) set in the biosafety cabinet; 96-well compound plates (Thermo Scientific, cat. no. 249946); 15-ml conical tubes (Thermo Scientific, cat. no. 339650); OT-2 pipetting robot (Opentrons, SKU 999-00111, 999-00002, or 999-00002); OT-2 accessories and tips: HEPA module (Opentrons, model GEN1), P300 GEN2 multichannel pipette (Opentrons). P20 GEN2 multichannel pipette (Opentrons), 15-ml reservoir (12-well, Opentrons, SKU 999-00076, or Nest, cat. no. 360112: or similar), 96 tips/box and refills (20 μl and 200 μl or 300 μl); Ultrasonic bath (Fisher Scientific, ca. no. 15336130; model: Branson 1800), or similar; Sonicator (Fisher Scientific, model: FB50); and ArrayScan™ XTI high-content analysis (HCA) Reader (Thermo Scientific), and associated image analysis software (Thermo Scientific HCS Studio).

### Day 1: Cell Plating

1. Culture the cells in the T-75 flasks with 10 ml maintenance medium per flask, until they are 70% to 90% confluent, changing the medium every 2-3 days depending on cell density.

Use two flasks for each cell line, one for the assay, and one as a backup and for passaging. 2. Pre-warm the assay medium and TrypLE to 22° C.-37° C.

3. In the biological safety cabinet, gently remove the medium from the cells in the T-75 flasks and gently rinse the cells twice with 5-10 ml DPBS per flask.

4. Immediately after removing the second DPBS rinse, coat the cells with 2 ml TrypLE per flask.

5. Replace the lids on the flasks and return the cells to the incubator (37° C., 5% CO₂) for 5-10 minutes.

Do not leave cells in TrypLE for more than 15 minutes to avoid over-digesting the cell membrane proteins.

6. After the 5-10-minute incubation period, add 4 ml assay medium to each flask and mix so that the fetal bovine serum (FBS) in the medium inactivates the TrypLE. Use the resuspension to rinse any remaining cells off the bottom surface of the flask, then stand the flask up on its end (opposite the cap) to pool the resuspension over a smaller surface area.

7. Mix the cells (gently and carefully; avoid making bubbles) ten times with a 10 ml serological pipette.

At this point, if detaching multiple flasks of a single cell type, cells of the same type may be pooled together into a single resuspension volume.

8. Remove 11 μl cell resuspension into a 2-ml microcentrifuge tube for counting.

9. Add 11 μl 0.4% trypan blue solution to the cell sample and gently mix with pipetting three times.

10. Transfer 10 μl dyed sample into each chamber on the cell counting slide, load the slide into the cell counter, and read the cell count and viability. Make sure the viability of the cells to be used for the assay is >90%.

11. In a 50-ml conical tube, prepare 35 ml cell solution for plating one 384-well plate (prepare 20 ml more for each additional cell plate if needed), at the concentration needed to achieve the desired number of cells per well. Plating volume for the 384-well plate is at 45 μl/well; HMC3 cells are prepared at 13,333 cells/ml to yield 600 cells/45 μl/well, and BV2 cells at 8,889 cells/ml to yield 400 cells/45 μl/well; and mouse primary microglia at 44,444 cells/ml to yield 2,000 cells/45 μl/well. Each 384-well plate can test 16 different treatments in duplicate in 10-point concentration serial dilutions. Plate the number of plates for an experiment accordingly.

12. Prepare the following in separate 50-ml conical tubes for washing tubes of the 384 Multidrop (or similar instrument can be used for plating cells into 384-well plates): >30 ml sterile water; 25 ml serum-free medium (this is the volume needed for plating 2 cell lines; add 10 ml per additional cell line); and 15-20 ml 70% ethanol.

13. Prepare the Multidrop and set it to dispense 45 μl/well into all the wells of a 384-well plate.

14. Label an empty clear-bottomed 384-well plate and set it on the plate carrier of the Multidrop. Remove the lid and set it aside.

15. Run ˜10 ml of serum-free medium through the instrument into a waste container.

16. Draw up some air into the tubing, then gently mix the first 50-ml conical tube of cells for plating. Draw up the cell suspension into the instrument. Gently agitate the cell suspension constantly, until plating is completed.

Ensure that no air bubbles are being drawn up. Be sure to have at least 22.5 ml cell suspension in the 50-ml tube for plating a 384-well plate with 45 μl well.

17. Initiate the cell plating program to plate the cells.

18. If plating multiple plates of the same cell type, be sure to add more cell suspension to the 50-ml conical tube, then repeat the plating.

19. Check the plate visually for any obvious volume errors, which can be corrected manually. 20. When the plating step is completed, empty the tubes of residual cell suspension.

21. Prepare tubes for plating the next cell line by sequentially running 20 ml sterile water followed by 10 ml serum-free medium through the tubes, then hold the tubes up in the air and draw up some air.

22. Repeat steps 16-19 with the second cell line.

23. Check the plates under a microscope to ensure that the cells were plated evenly and with no visible contamination.

24. Clean tubes by sequentially by running 30 ml sterile water through the instrument followed by 10 ml 70% ethanol until all the ethanol has run through into the waste container.

25. Power off the instrument. Clean and store the instrument and accessories.

26. Incubate plated cells overnight (16-24 hours) at 37° C. with 5% CO₂ before treatment.

### Day 2: Compound Serial Dilution and Treating Cells

The following procedures are for a single plate of each cell type, with duplicates of each compound dilution series within each plate.

Protocols for the OT-2 (or other similar liquid handling) robot (compound dilution protocol and compound distribution protocol) will need to be designed and tested before proceeding. Multichannel pipettes in the robot are oriented along the Y (e.g., plate column) axis using the protocols.

27. Use a 96-well compound plate for serial dilutions. Start by manually filling column 2 wells with 97 μl assay medium, then add 3 μl 20 mM compound in DMSO in each well to get 600 UM and 3% dimethyl sulfoxide (DMSO) of each compound as 10×.

28. Make 3% DMSO in assay medium (30 μl DMSO and 970 μl medium/ml), prepare 6 ml for each compound plate, add the 3% DMSO-medium at 60 μl/well to wells in column 3-12.

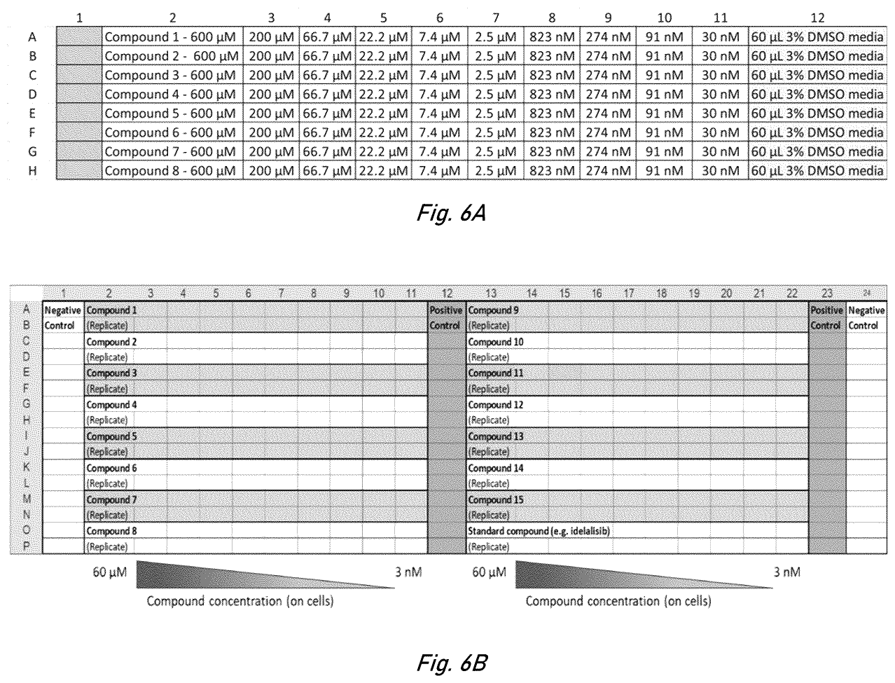

29. Prepare a 1:3 dilution series by sequential transferring/mixing (three times) 30 μl from each well to the next well across the rows, from column 2 to column 11. Column 12 will remain without compound, as the vehicle control column. An example plate map of compound dilution is shown in FIG. 6A.

30. Transfer and position the compound plates, cell plates, and a box of 20 μl tips securely in the proper positions within the robot enclosure, according to the protocol setup on the robot app/software. Remove any lids or covers from these items.

31. Use the 8-channel pipette to pick up 20 μl control DMSO-medium from column 12, and transfer it to columns 1, 12, 23, and 24 in each cell plate, distributing 5 μl/well, twice per column since it is a 96-well to 384-well transfer. Change tips after this step.

32. Pick up 10 μl compounds from column 2 of the compound plate, transfer to column 2 of a 384-well cell plate through two duplicate actions (5 μl/well, 2 distributions/column); repeat these actions (pick up and transfer) through column 3-11 to treat the left half of the 384-well cell plate.

33. Repeat step 32 with a new set of tips and the second compound plate to treat the right half (column 13-22) of the 384-well cell plate.

34. Return cell plates to the incubator at 37° C. with 5% CO₂ for 24 hours before proceeding with the next step.

An example map of treated cell plate is shown in FIG. 6B.

The above compound distribution procedures are performed with the OT-2 automatic pipetting robot; These steps can be done with similar instruments. The compound distribution protocol requires the 20 μl 8-channel pipette to transfer 5 μl of each concentration of each compound from the 96-well compound plate to the corresponding wells in the 384-well cell plates, changing tips for each compound plate.

Day 3: pHrodo™-Myelin/Membrane Debris Seeding

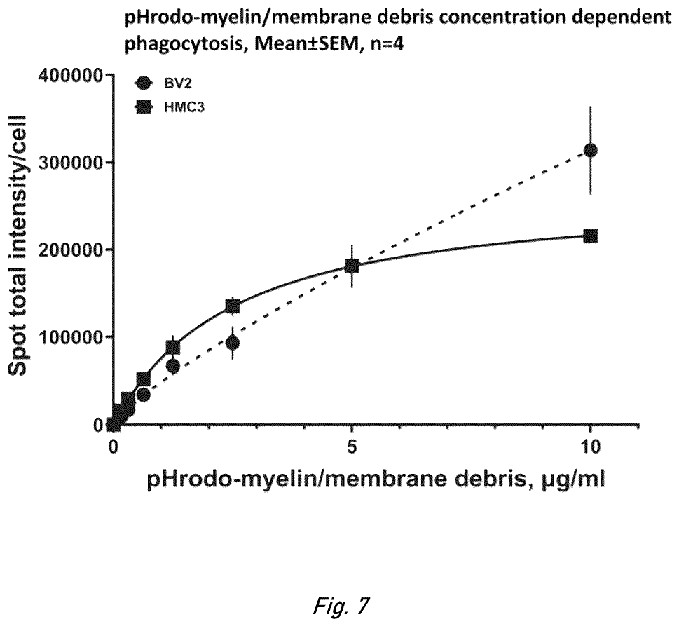

The pHrodo™-myelin/cell debris seeding protocol uses the 20 μl 8-channel pipette to transfer 5 μl pHrodo™-myelin/membrane debris from the compound plate to the corresponding wells in the cell plates so that final cell exposure to 5 μg/ml that was experimentally determined (FIG. 7), changing tips after every transfer (from the compound plate to the cell plate) to avoid potential cross-contamination of compounds into the pHrodo™-myelin/cell debris in the compound plate. The following protocol is for two cell plates; adjust volumes accordingly if using additional plates.

35. Pre-warm 5 ml cell culture assay medium to 37° C.

36. For each cell plate, thaw 2 aliquots of pHrodo™-labeled myelin/cell debris (ea. 55 μl at 1 mg/ml) in the water bath at room temperature.

37. Vortex the aliquots briefly, then sonicate them in the sonicating water bath for 2 min, vortex the aliquots again, and transfer them into the biosafety cabinet.

38. In the biosafety cabinet, dilute each pHrodo™-myelin/membrane debris aliquot with 1.1 ml assay medium (yielding 50 μg/ml pHrodo™-myelin/membrane debris), pool the four aliquot dilutions in a 15-ml conical tube, and mix thoroughly.

39. Distribute 260 μl/well diluted pHrodo™-myelin/membrane debris to wells in the first two columns (one column per cell plate) of a clean compound plate for seeding cells with the OT-2.

40. Use 20 μl tips to transfer 5 μl/well of the pHrodo™-myelin/membrane debris from the compound plate to two 384-well cell plates; seed control columns 12 and 23 first, changing tips after every seeding to avoid cross contamination among wells when seeding wells in columns 2-11 and 13-22. Total seeded wells cover columns 2-23 of a 384-well cell plate; leave wells in columns 1 and 24 without seeding as negative controls.

41. Return cell plates to the incubator at 37° C. with 5% CO₂ for 20 hours before next step.

### Day 4: Nuclear Staining and Imaging

The following protocol is for two cell plates; adjust volumes accordingly if using additional plates.

42. Perform nuclear staining as follows:

Pre-warm 22 ml of assay medium to room temperature.

Add 22 ul of 10 mg/ml Hocchst 33342 into 22 ml assay medium; mix well.

Use 200 μl tips to transfer 20 μl/well of the diluted Hoechst 33342 to 384-well cell plates, final cell exposure to 2.6 μg/ml Hoechst 33342; return cell plates into incubator for 30 min at 37° C. with 5% CO₂ for incubation.

This step is performed with the OT-2 automatic pipetting robot but can be done with similar instruments.

Remove cell plates from the incubator for imaging.

43. Perform imaging as follows:

Power on the ArrayScan™ XTI high-content imaging instrument, start Thermo Scientific HCS Studio Cell Analysis Software, choose the template of General Spot Measurement Tool. Set two channels, channel-1 for nuclear fluorescence (Hoechst 33342, ex. 386 nm, em. 440 nm) and channel-2 for phagocytosis vesicle fluorescence (pHrodo™ red, ex. 560 nm, cm. >585 nm).

The plate imaging alignment may need to be optimized on the instrument at this time if not properly centered on wells.

Load the plate and optimize the imaging settings using the 10× objective, adjust appropriate imaging settings for each cell type to be imaged including automatic focusing on nucleus, find optimal exposure time for each channel, set the software to take a z-stack of 3 images with 6.7 μm apart to generate a projected image for analysis and taking 4 projected images/well.

Take example images from max and min wells and analyze these images through image analysis process: 1) segment cells on nucleus to identify individual cells; 2) expand identified edge of nucleus 30-50 μm to cover whole cell area: 3) identify phagocytosis vesicles in cell area for analysis.

Start the imaging, collecting four images per well. For analysis, collect cell counts per well, average nuclear intensity, and mean total phagocytosis spot intensity/cell, plus any other additional parameters of interest, for quantitation.

In the ArrayScan™ system, choose the template of “General Spot Measurement Tool” that allows to identify and analyze both the nuclei and the phagocytosed vesicles. A separate analysis protocol is needed for each cell type since different cell sizes and morphology need to be optimized for each specific cell type before starting the automated analysis. The imaging and analysis can be done with other high-content imaging systems using the similar analysis method.

Support Protocol: PROCEDURES TO ISOLATE MYELIN/MEMBRANE DEBRIS from Mouse Brain and Label with Phrodo™

When developing the phagocytosis assay, we questioned what would be a proper ligand for measuring microglial phagocytosis. There are many options from which to choose, such as commercially available recombinant Aβ, Tau, microbeads, zymosan, and bacteria, or experimentally isolated dead neurons, synaptosomes or myelin from mouse brains. Since our project targets the TREM2 pathway, and since myelin/membrane debris has been shown to be a good ligand for TREM2 binding (Andreone et al., 2020; Ennerfelt et al., 2022; Nugent et al., 2020) and a natural material in the brain that is cleaned up by microglia in both homeostasis and neurodegenerative diseases, we decided to use myelin/membrane debris for the microglial phagocytosis/cell health assay. Isolation of crude myelin/membrane debris from 6-8 week old wild type C57BL/6J mouse brains was followed and modified on established literature protocols using sucrose gradients (0.32 M/0.85 M) and ultracentrifugation (Andreone et al., 2020; Erwig et al., 2019). The crude myelin/membrane debris was diluted to 1 mg/ml (protein equivalent) in DPBS, stored in a −80° C. freezer, and subsequently labeled with pHrodo™ red dye.

### Additional Materials

6-8 week old C57BL/6J mice (B6; The Jackson Laboratory, 000664), 3 mice for each preparation; Anesthesia (see recipe infra); Ultrapure distilled water (Invitrogen cat. no. 10977-015); or autoclaved MilliQ water; Sucrose (Thermo Scientific, cat. no. A1558336) for preparing 0.3 M, 0.32 M and 0.85 M solutions in distilled water. 20 ml each. keep on ice; Lysis buffer (see recipe infra); Pierce Rapid Gold BCA Protein assay kit (Thermo Scientific, cat. no. A53226); pHrodo™ red, 10 mM (see recipe infra); NaHCO₃, 1M (see recipe infra); pH 4 buffer (Fisher Scientific. cat. no. Orinon 910104); Glass petri dish (Fisher Scientific, cat. no. 08749B); Razor blades; Glass dounce homogenizer (Fisher Scientific, cat. no. 06-435B); 5-ml ultracentrifuge tubes (SETON Scientific, cat. no. 5022) or similar 5-10 ml tubes for ultracentrifuge; Sterilized Pasteur pipette; Beckman Optima XPN-80 ultracentrifuge and SW 40 Ti rotor, 4° C. (or similar); Eppendorf centrifuge 5424R, 4° C.; Microcentrifuge (BT labsystems, model. BT604); Benchtop shaker (Fisher Scientific, cat. No. 88861045); and 96-well clear-bottom cell culture plates (Thermo Scientific, cat. no. 165305).

Anesthetize 6-8-week-old C57BL/6J mice (up to 3 mice for each prep) with anesthesia through intraperitoneal (ip) injection of 20 μl/gram body weight, then euthanize by cervical dislocation. (All experiments were approved by the Institutional Animal Care and Use Committee at Indiana University).

Remove brain tissues and place each brain in 1.2 ml cold lysis buffer in a glass petri dish on ice, cut into fine pieces with a razor blade and then dissociate via dounce homogenization (20× loose, then 20× tight).

Transfer the homogenate to 5-ml ultracentrifuge tubes with 1.2 ml/tube. Weigh loaded tubes to have balanced pairs for ultracentrifugation.

Add 1.2 ml of 0.32 M sucrose carefully underneath with a sterilized Pasteur pipette, and then add 1.2 ml of 0.85 M sucrose underneath both layers.

Precool the Beckman Optima XPN-80 ultracentrifuge to 4° C., load samples, and spin 30 min at 75,000×g, 4° C. (in an SW 40 Ti rotor, or similar) with low acceleration and deceleration.

Carefully remove myelin at the 0.32 M/0.85 M sucrose interface from centrifuge tubes and resuspended in 10 ml cold distilled water in a 15 ml tube, mix well.

Some tubes may fail to generate the myelin layer between 0.32 M/0.85 M sucrose gradients; we collected about 120 μl from the top of the whitish precipitates (about top 10% of the precipitates) which are membrane debris.

7. Transfer the myelin/membrane debris to 2-ml microcentrifuge tubes with 1.2 ml/tube.

8. In a microcentrifuge (Eppendorf centrifuge 5424R), spin 10 min at 14,000×g, 4° C., with maximum acceleration and deceleration; discard supernatant, resuspend pellets in 1.2 ml cold distilled water for each tube and vortex. Repeat three more times; after final centrifugation step, resuspend myelin/membrane debris with DPBS at 1 ml/tube.

9. Combine myelin from the 2-ml tubes into one 15-ml tube on ice for sonication at max amplitude for 15 seconds, sonicate three times.

10. Perform BCA assay to determine protein concentration, then dilute with DPBS to 1 mg/ml protein concentration, make 5 ml/tube aliquots (using 15-ml tubes) store in −20° C. or −80° C. freezer, or process for pHrodo™ labeling immediately through the following procedures.

11. Transfer 15 ml thawed 1 mg/ml myelin/membrane debris into a 50-ml conical tube.

12. Add 1.65 ml 1 M NaHCO₃ to the 15 ml myelin/membrane debris. Mix by pipetting.

13. Add 150 μl 10 mM pHrodo™ red in DMSO to the myelin/membrane debris in the 50-ml conical tube. Mix by pipetting.

14. React for 45 min at room temperature: wrap the 50-ml tube with aluminum foil and set the tube in a rack on a shaker with gentle shaking.

15. Remove unbound dye as follows:

Transfer labeled myelin/membrane debris from the 50-ml tube to about 10-12 2-ml centrifuge tubes with 1.5 ml/vial (weigh tubes for balance before centrifuging).

Spin 10 min at 14,000×g. 4° C., using the Eppendorf centrifuge 5424R.

Discard supernatant.

Resuspend labeled myelin/membrane debris in cold DPBS (keep on ice) by vortexing.

Repeat b-d three more times to remove unbound pHrodo™ red dye

16. Resuspend the labeled myelin/membrane debris in cold DPBS to total 15 ml (same as original volume of the unlabeled myelin, to keep the concentration at about 1 mg/ml); pool in a 50-ml conical tube on ice.

17. Sonicate the labeled myelin/membrane debris solution on ice (put the sonicator probe into the myelin solution in the 50-ml tube kept on ice in a beaker) at max strength for 15 s; perform this step three times.

18. Make aliquots of 55 μl/vial in 2 ml vials (two aliquots are required for each 384-well plate for one assay); store at −20° C. (up to 3 months) or −80° C. (up to 1 year) or use immediately. The final molar ratio of pHrodo™ red/myelin is at least 2 (counting myelin basal protein with MW 20 kD as the average MW of the sample).

19. Check the quality of the pHrodo™-myelin/membrane debris prep with a fluorescent microscope as follows:

Prepare pHrodo™-myelin/membrane debris at 5 μg/ml in DPBS (1:200 dilution of 1 mg/ml stock with DPBS).

Transfer 100 μl of the prep to a well of a 96-well clear-bottom cell culture plate.

Image with a fluorescent microscope using a 20× lens to get both transmitted light and fluorescent (ex. 560, em. 585 nm) images.

When in DPBS (about pH 7), the sample should show little or no fluorescence.

Add 200 μl of a pH 4 buffer to the well and repeat step c.

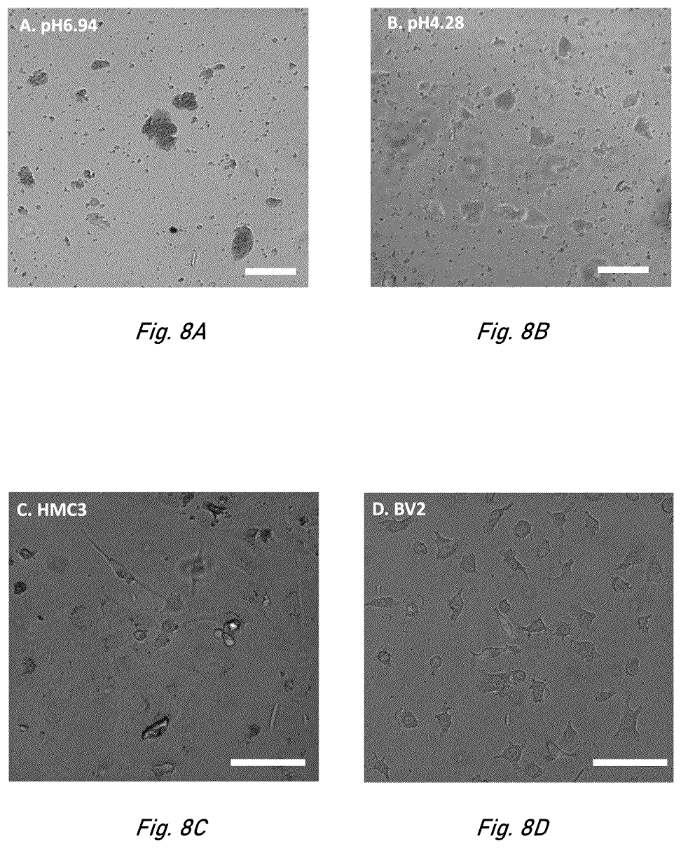

The pHrodo™-myelin/membrane debris should show strong red fluorescence in acid solution (around pH 4.2) (FIGS. 8A and 8B).

20. Check pHrodo™-myelin/membrane debris signal in phagocytosis as follows:

Plate cells in a clear-bottomed microplate (1.000 cells/45 μl/well for a 384 well plate, or 5,000 cells/90 μl/well for a 96-well plate).

Incubate overnight in an incubator at 37° C. with 5% CO₂.

On day 2, prepare a 1:20 dilution (as 10× for seeding to cells) of the 1 mg/ml pHrodo™-myelin/membrane debris (e.g., 5 μl mixed with 95 μl medium),

Add the diluted pHrodo™-myelin/membrane debris into the cell plate (e.g., 5 μl/well for a 384-well plate with 45 μl cells/well plated, or 10 μl/well for a 96-well plate with 90 μl cells/well plated) so that cells are exposed to 5 μg/ml pHrodo™-myelin/membrane debris.

Incubate at 37° C. with 5% CO₂ for 4-5 hr.

Check phagocytosis with a fluorescent microscope as above in step 19 (FIGS. 8C and 8D).

Using 5 μg/ml pHrodo™-myelin/membrane debris for the assay was determined with concentration dependent phagocytosis on both HMC3 and BV2 cells (FIG. 7).

### Reagents and Solutions

### Anesthesia

1.25 g 2,2,2-tribromoethanol (ACROS organics cat. No. 421430500); 2.5 ml amylene hydrate (ACROS organics cat. no. 166620010); Dissolve by heating to 40° C. with stirring; Add distilled water to total volume of 100 ml; Filter through 5-micron filter; and aliquot and cover with foil. Store at 4° C. and use before the expiration date or when pH reaches >5.0.

### Assay Medium

500 ml Dulbecco's modified eagle medium (DMEM), high glucose; GlutaMAX supplement, HEPES (ThermoFisher Scientific, cat. no. 10564-011); 50 ml Corning regular fetal bovine serum (Fisher Scientific, cat. no. MT35010CV); 5 ml penicillin/streptomycin (Sigma-Aldrich, cat. no. P4333, with 10,000 U penicillin and 10 mg streptomycin/ml); and filter through disposable PES filter units (Fisher Scientific, cat. no. FB12566504). Store up to 4 weeks at 4° C.

### Lysis Buffer

### Prepare 10 ml:

10 mM HEPES; 5 mM EDTA 0.3 M sucrose; and supplement with protease inhibitor (Roche, 04-693-159-001) or similar. Prepare fresh before use and store on ice.

### Maintenance Medium

500 ml DMEM, high glucose, GlutaMAX supplement (ThermoFisher Scientific, cat. no. 10566-016); 50 ml Corning regular FBS (Fisher Scientific, cat. no. MT35010CV); 5 ml penicillin/streptomycin (Sigma-Aldrich, cat. no. P4333, with 10,000 units penicillin and 10 mg streptomycin/ml); and filter through disposable PES filter units (Fisher Scientific, cat. no. FB12566504). Store up to 4 weeks at 4° C.

### NaHCO₃, 1 M

168 mg NaHCO₃(MW 84.01) (Fisher Scientific, cat. no. S233-500) and 2 ml sterile MilliQ water. Store at −20° C. up to 6 months.

pHrodo™ Red, 10 mM

pHrodo™ red from Thermo Fisher Scientific is a fluorogenic, cell-permeant pH indicator that is non-fluorescent at a neutral pH and increases in intensity with an increase in acidity involved in endocytosis and phagocytosis. pHrodo™ is often used to label proteins or biomolecules to track this process. pHrodo™ Red has an excitation peak at 560 nm and an emission peak at 585 nm, making it useful in both flow cytometry and fluorescence microscopy.

1 mg pHrodo™ red, warmed to room temperature and spun briefly with a microcentrifuge before opening (1 mg vial, Thermo Fisher, cat. No. P36600); 150 μl DMSO, ≥99.9% (Sigma-Aldrich, cat. no. 276855-100 ml); vortex and mix with pipetting to ensure that the dye is well dissolved; and prepare fresh immediately before using.

### DISCUSSION

### Background Information

Microglial phagocytosis has been studied for its role in brain development (Cunningham et al., 2013; Matcovitch-Natan et al., 2016; Wake et al., 2013) and maintenance of homeostasis through surveillance and cleaning of cell debris, including dead cells and abnormal protein aggregates (Li & Barres, 2018; Mundt et al., 2022; Nimmerjahn et al., 2005; Salter & Stevens, 2017; Streit et al., 2004). Drug discovery targeting microglial phagocytosis function has been proposed as a therapeutic strategy for Alzheimer's disease and other neurodegenerative diseases (Nizami et al., 2019; Salter & Stevens, 2017). A reliable cellular assay capable of measuring drug-regulated phagocytosis activity while distinguishing that activity from phagocytosis modulation caused by cell stress is needed for drug discovery research targeting microglial phagocytosis. This need motivated us to develop and establish a high-content microglial phagocytosis/cell health assay to meet the challenge.

The primary advantage of the assay is the capability to measure phagocytosis and cell health simultaneously, allowing for the discernment of drug efficacy on phagocytosis (either stimulation or inhibition) in the absence of cell stress, since microglia under stress can show either higher or lower phagocytosis than that of resting microglia, as we have repeatedly observed. The multiplex feature of the assay has advantages over assays utilizing flow cytometry or imaging techniques to measure only phagocytosis alone. In addition, the assay with 384-well plates has the capacity required for screening or supporting molecule structure-activity-relationship studies for drug discovery projects. Another distinguishing feature of the assay is that it does not have washing steps and is a mix-and-read assay. Using pHrodo™ (an acid-activated fluorophore)-labeled myelin/membrane debris is the key to avoid any washing step for measuring cellular phagocytosis in the assay. Only live cells have bright phagocytosis vesicles with well-maintained inner acidic pH, and these are easily distinguished from extracellular pHrodo™-myelin/membrane debris which shows negligible or low signal in a neutral pH environment. The washing-free protocol makes the cell count/well a reliable measurement for the evaluation of cell proliferation and viability under drug treatments (>24 hr), which combined with the measurement of nuclear intensity allowed estimation of treatment induced apoptosis. We think that combined measurements of cell count and nuclear intensity allow estimating overall status of cell population under experimental treatments, named them cell health measurements. One of the limitations of the 384-well plate assay is that it requires the use of an automatic pipetting instrument for liquid additions since it is difficult to keep uniformity of 384-well plates with manual operations. However, the assay can be performed with 96-well or other cell plates, through manual operations, to meet individual research needs. Another limitation is the necessity of an automatic high-content imaging system to handle the large numbers of images and analyses. The assay can be done manually as well if not used for drug discovery screening projects, by imaging with a common fluorescent microscope and performing image analysis with a proper software.

### Critical Parameters

### Cells

It is critical to check the cell viabilities when starting the assay. When plating cells, we check to ensure that viability is always >90%. Plating densities need to be experimentally determined and kept consistent to make the assay reproducible. The assay medium contains HEPES to maintain pH since during imaging the cell plates will be out of the incubator and exposed to room temperature and environmental air. We established this assay with microglial cell lines, HMC3 (immortalized human microglia) and BV2 (immortalized mouse microglia), and we have also used it with mouse primary microglia. The protocol can be used for other cells as well, such as macrophages and iPSC-derived microglia, to measure phagocytosis and cell heath simultaneously.

### Ligands

The pHrodo™-myelin/membrane debris was chosen as the ligand for our studies targeting TREM2 pathway because TREM2 has been shown to bind membrane lipids, especially the phosphatidylserine (PS) of dead cells. The protocol can be used to study the phagocytosis of other ligands such as synaptosomes, zymosan, bacteria, protein aggregates (Aβ, Tau), and microbeads, to suit individual research purposes. The seeding concentration and incubation time need to be experimentally established and kept consistent for reproducibility. Choosing 20-24 hours as the pHrodo™-myelin incubation time was based on our observation that maximal phagocytosis took 4 hours and remained maximal for 24 hours, so that maximal phagocytosis can be measured. For experiments aiming to measure the initial phase of phagocytosis, the time course of phagocytosis needs to be experimentally determined.

### Imaging

Collecting well-focused images is critical for quantitative high-content analysis since out-of-focus images always lead to inaccurate quantitative measurements. We set up automatic imaging using 3 focus planes separated by 6.7 μm along the Z-axis for every field, then save a single projected image formed from those 3 images for analysis. This is critical to ensure quality of the quantitative imaging analysis for subcellular measurements.

### Troubleshooting

Troubleshooting steps are presented in Table 2.

TABLE 2
Troubleshooting Guide for the Assay

| Problem | Possible Cause | Solution |
|---|---|---|
| Poor | Poor cell viability | Make sure cell viability |
| phagocytosis | | >90%; cell plating density |
| signal | | may be too high, check if |
| | | medium turned yellow at the |
| | | end, then adjust cell plating |
| | | density accordingly |
| Highly | 1. Uneven cell plating, | 1. Mix cell suspension well |
| variable data | drug or pHrodo ™-myelin | before plating; make sure the |
| | addition to wells; | multidrop and automatic |
| | | liquid handler is in normal |
| | | condition; |
| | 2. Imaging was out of | 2. Make sure the proper |
| | focus; and/or | image projection of 3 focal |
| | | planes is used; control wells |
| | | should be uniform; |
| | 3. Max and min signals | 3. Check imaging setting and |
| | were not set properly. | signal dynamic in range for |
| | | covering max and min. |

### Data Analysis

All collected raw data are normalized to the untreated control with following formula: % of control=100×(X−Min)/(Max−Min). Factors in the formula are: X is raw data from a treated well, Max is an average measurement from pHrodo™-myelin seeded but untreated wells, and Min is an average measurement from no pHrodo™-myelin untreated wells. Normalized data are used for curve fitting that is done with the variable slope (four parameters) using the Prism software.

### Understanding Results

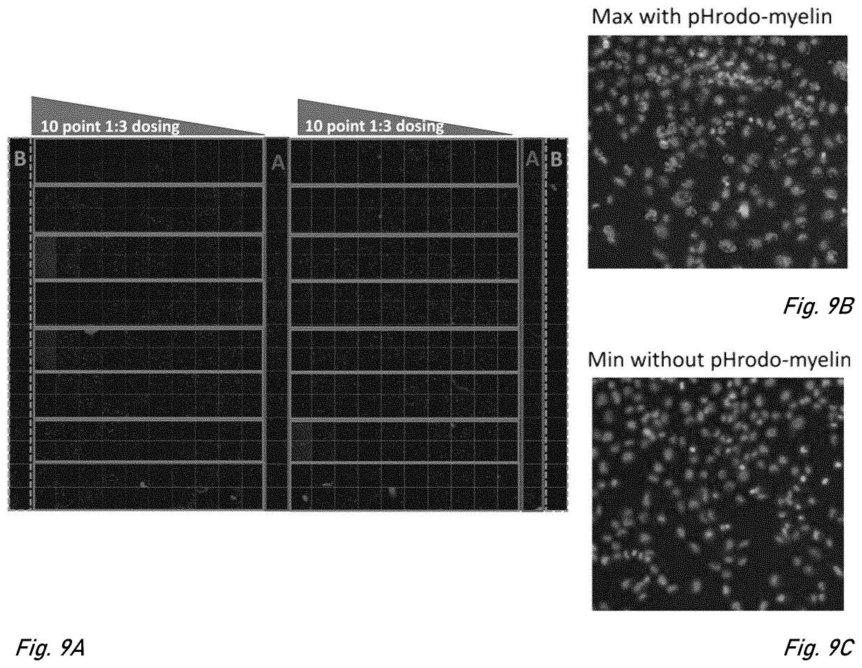

The 384-well plate assay design is shown in FIG. 9A-9C. Each plate contains two Min (negative) control columns (columns 1 & 24, without pHrodo™-myelin seeding) and two Max (positive) (FIG. 9B) control columns (columns 12 & 23, with pHrodo™-myelin) (FIG. 9C) so that these controls come from edge and middle wells, respectively. The rest of the wells are used for testing compounds in 10-point concentration response, in duplicates for each compound; a total of 16 compounds can be tested in one plate.

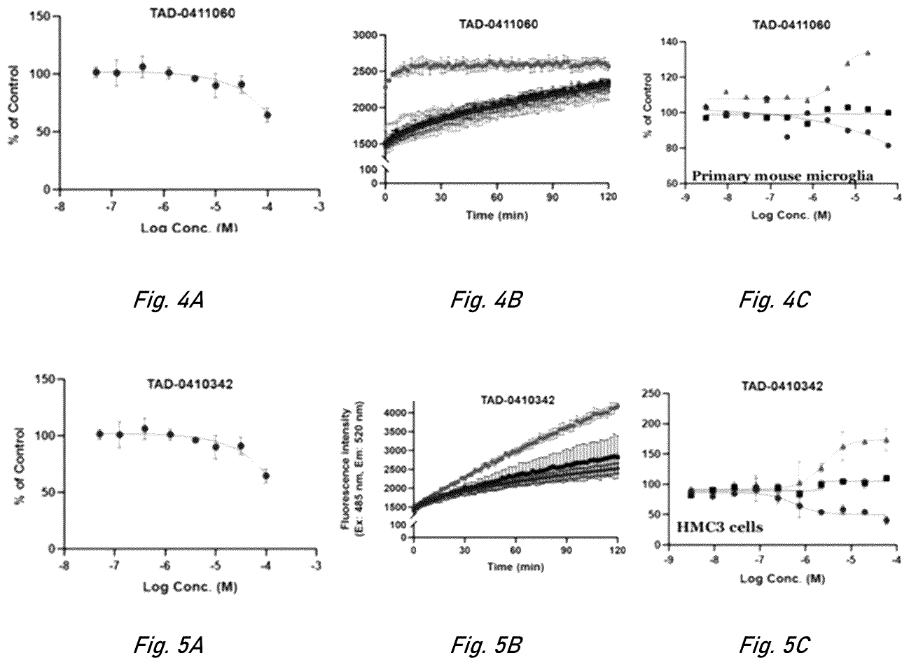

To ensure that the measurements reflect the steady state of compound treatment effects and cell health, the assay was set with a total compound treatment time of 48 hr, including pHrodo™-myelin/membrane debris seeding performed 20-24 hr after compound addition. FIG. 4 shows the image analysis process to identify individual cells and phagocytosis vesicles. Among many available quantitative measurements from the image analysis, we selected three parameters for the assay:

1) The mean total intensity of phagocytosis vesicles/cell—to measure phagocytosis. There are multiple parameters generated from the image analysis, including phagocytosis vesicle area, number, and various fluorescence intensity measurements. Comparing these parameters, the total fluorescence intensity of vesicles/cell is the most sensitive one and was chosen for quantifying phagocytosis.

2) The total cell counts—to measure overall cell viability, which is affected by proliferation and cell death. We chose to take 4 images/well (with the 10× objective lens) that cover about 50% of the total area of a well in a 384-well plate to represent cell count of the well.

3) The average nuclear intensity—to measure drug-induced apoptosis, with high signal indicating nuclear condescension (a sign of early apoptosis) and low signal indicating DNA degradation (a sign of later apoptosis, when activated caspases digest DNA).

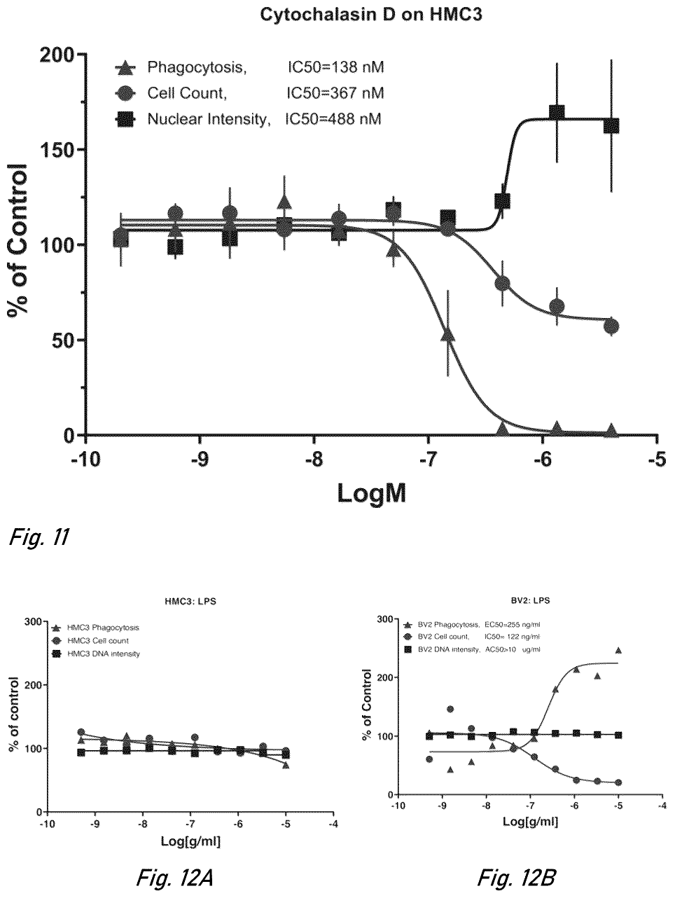

When evaluating the effects of a treatment, the combination of the three measurements allows for the determination of treatment effects on phagocytosis and of treatment caused cytotoxicity. We tested cytochalasin D in the assay, for its known effect of blocking actin filament formation (Casella et al., 1981), thus inhibiting phagocytosis and other actin-mediated cell activities (Meena et al., 2021; Melzer et al., 2019; Murai et al., 1993; Tilney & Portnoy, 1989; Vance et al., 2016). We observed that 24-hr cytochalasin D treatment (FIG. 11) inhibited HMC3 cell phagocytosis with IC₅₀=138 nM, with lower potencies for its effects on cell count (IC₅₀=367 nM) and nuclear intensity (EC₅₀=488 nM). These results indicated that cytochalasin D inhibited phagocytosis in HMC3 cells, with cytotoxicity effects indicated by a cell number decrease and a concurrent nuclear intensity increase. It has been shown that cytochalasin D inhibits cell division at the end of the mitotic phase of the cell cycle and causes cells to round up (Margadant et al., 2013); which can explain our observation of decreased cell counts and increased nuclear intensity in HMC3 cells. When testing LPS on the HMC3 and BV2 microglia, results showed that HMC3 cells did not respond to LPS (FIG. 12A), while BV2 cells showed stimulation of phagocytosis parallel with decreased cell count, both in concentration-dependent responses, and no nuclear intensity changes up to 10 μg/ml LPS (FIG. 12B). The experiment was performed at least two times. These experimental results validated the assay performance. Observations that HMC3 cells failed to respond to the LPS suggested that HMC3 cells at basal status might lack Toll-like receptor 4 (TLR4) or related signaling molecules since LPS is known to bind to Toll-like receptor 4 (TLR4), thus stimulating immune cells. Different responses from different cell lines are not uncommon, and the mechanisms behind these differences remain to be studied.

To explore cellular signaling regulation of phagocytosis with the assay, we chose a highly selective human PI3Kd inhibitor, idelalisib (Somoza et al., 2015), as a reference compound, based on knowledge that PI3K regulates cellular phagocytosis (Desale & Chinnathambi, 2021; Gillooly et al., 2001). Idelalisib (48-hr treatment) inhibited HMC3 cell phagocytosis in a concentration-dependent manner with an IC₅₀ around 500 nM, with no significant cell count decrease or nuclear intensity changes (FIG. 13A). In comparison, idelalisib did not inhibit phagocytosis in BV2 cells, but increased phagocytosis in parallel with decreasing cell counts and increasing nuclear intensity, suggesting that the stimulation of phagocytosis was related to cell stress (FIG. 13B). These results were very reproducible, with similar results observed in more than 30 repeated assays, or a 100% success rate.

Saracatinib (AZD0530), an inhibitor of the Src/abl family of tyrosine-kinase including Fyn and Lyn, has been repurposed for potential AD therapy and has been in clinical trials (Nygaard, 2018; Nygaard et al., 2015; van Dyck et al., 2019) since some Src kinases have been found to be involved in AD pathogenesis (Dhawan & Combs, 2012). When testing saracatinib in our assay, we observed that it was highly active in stimulating HMC3 cell phagocytosis in the concentration range without cytotoxicity, EC₅₀<200 nM (FIG. 14A graph and images). In comparison, BV2 cells responded to saracatinib differently, with no stimulated phagocytosis in the sub-μM concentration range, and when concentrations >1 μM showing cell count decreases and biphasic nuclear intensity changes (first increased then decreased), which indicate nuclear condensation followed by DNA degradation (FIG. 14B graph and images). Together, these results suggest that the phagocytosis increases under the saracatinib treatment as a stress reaction in BV2 cells. Similar results were observed in two repeat experiments. The mechanisms behind the different responses from the two microglial cell lines to saracatinib may be from possible differ in target structures in human versus mouse and remain to be studied. The observation that saracatinib significantly stimulates phagocytosis in HMC3 cells is interesting and may constitute an additional mechanism contributing to the therapeutic efficacy of the drug for AD.

Based on the results of testing idelalisib (inhibition effect) and saracatinib (stimulation effect), we showed that microglial phagocytosis of pHrodo™-myelin/membrane debris can be regulated by small molecule drugs; therefore, this assay can serve as a good in vitro model for drug discovery of small molecules targeting specific cellular signaling pathways with the aim to regulate the phagocytosis. We suggest that idelalisib and saracatinib can be used as references for the phagocytosis assay on microglia such as HMC3 cells. The assay was applied for the characterization of chemical probes targeting microglia, with results contributing to two papers (Potjewyd et al., 2022a, 2022b). Currently, the assay is being used in our AD drug discovery projects targeting the TREM2 pathway of microglia, specifically for the selection of INPP5D inhibitors and PLCG2 activators respectively. In conclusion, the microglial phagocytosis and cell health high-content assay provides a high-capacity tool measuring regulation of phagocytosis and cell health simultaneously, which can be helpful for drug discovery studies targeting the microglial phagocytosis.

### Time Considerations

Time considerations for the protocols are presented in Table 3.

TABLE 3
Time Considerations for Microglial Phagocytosis/Cell Health High
Content Assay

| | |
|---|---|
| Step | Time |

: Microglial phagocytosis/cell health high-content assay
Day 1

| | | |
|---|---|---|
| Detach and count cells | 20 | minutes |
| Plate cells | 1 | hour |

| | |
|---|---|
| Incubation | overnight |

Day 2

| | | |
|---|---|---|
| Compound plate preparation | 30 | minutes |
| Compound distribution to cells | 20 | minutes |

| | |
|---|---|
| Incubation | overnight |

Day 3

| | | |
|---|---|---|
| Prep pHrodo ™-myelin/membrane debris | 20 | minutes |
| Add pHrodo ™-myelin/membrane debris to cells | 20 | minutes |

| | |
|---|---|
| Incubation | overnight |

Day 4

| | | |
|---|---|---|
| Nuclear Staining | 45 | minutes |
| Imaging | 3 | hours |
| Data analysis | 3 | hours |

: Procedures to isolate myelin/membrane debris from mouse brain and
label with pHrodo ™

| repare solutions | 30 | minutes |
|---|---|---|
| Collect mouse brain tissue | 30 | minutes |
| Homogenize brain and prep for ultracentrifugation | 20 | minutes |
| Ultracentrifugation | 45 | minutes |
| Collect myelin/membrane debris from centrifuge tubes | 10 | minutes |
| Wash collected myelin/membrane debris | 60 | minutes |
| Sonicate collected myelin/membrane debris | 5 | minutes |
| BCA protein assay | 60 | minutes |
| Thaw and solubilize sample and reagents | 30 | minutes |
| Add dye and NaHCO₃ to myelin/membrane debris | 5 | minutes |
| Incubate in dark (on shaker) | 45 | minutes |
| Remove unbound dye | 60 | minutes |
| Resuspend and sonicate labeled myelin/membrane debris | 20 | minutes |
| Aliquot labeled myelin/membrane debris | 20 | minutes |
| Check prep quality in fluorescent microscope | 30 | minutes |
| Set cell culture for testing phagocytosis | overnight | |
| Check phagocytosis with cells | 5 | hours |

### Example 3

IUSM-Purdue TREAT-AD Center Target Enablement Resource m-3M3FBS Analogs Evaluated as PLCγ2 Chemical Probes

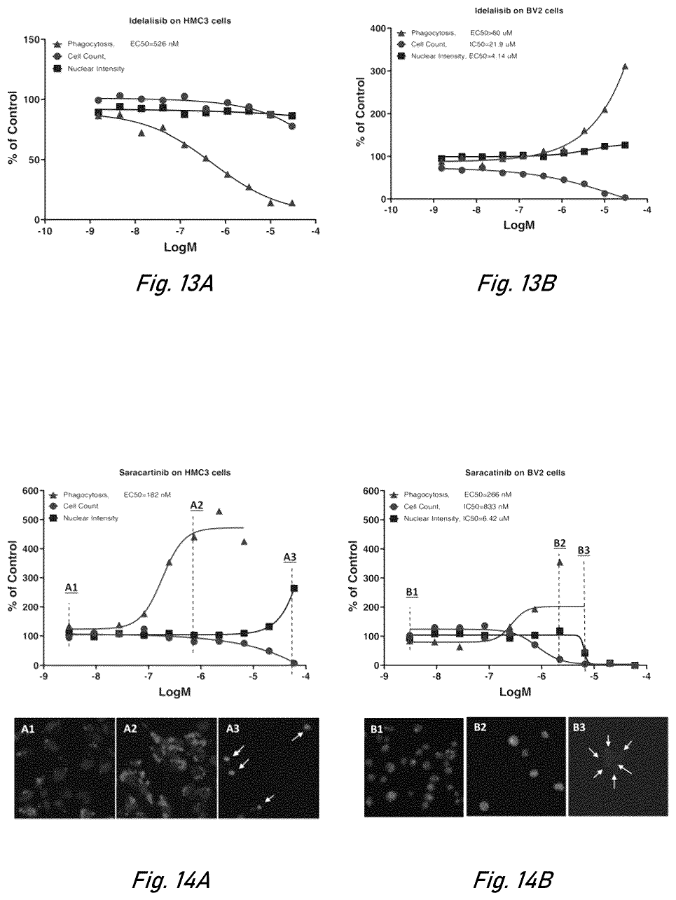

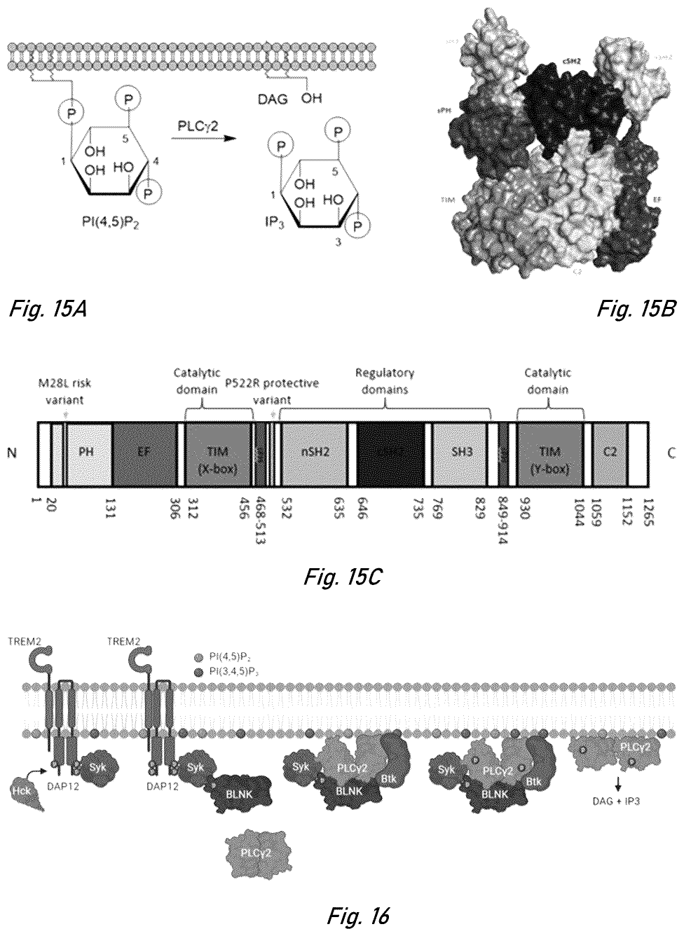

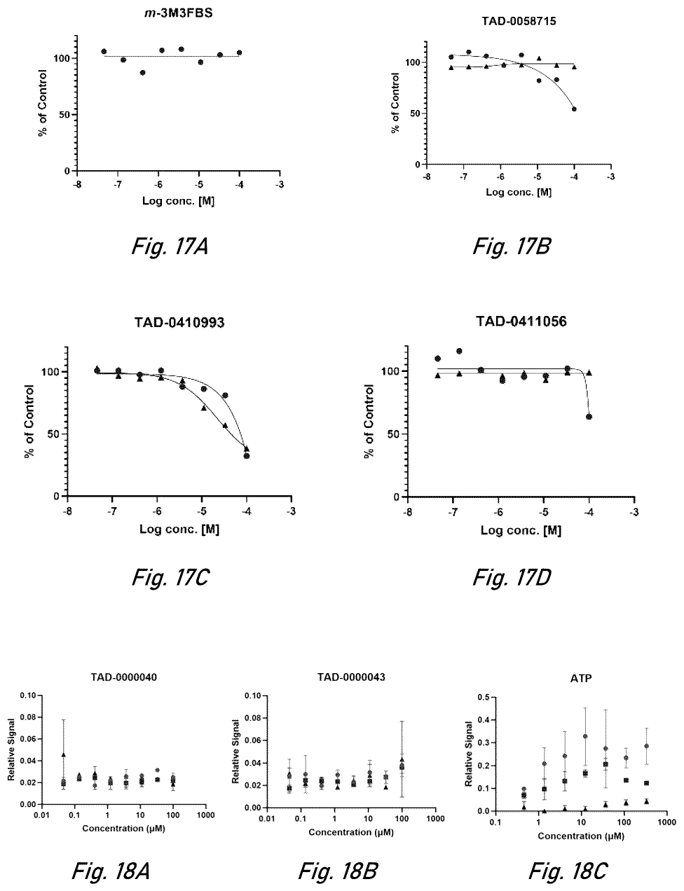

Genome-wide association studies (GWAS), whole-genome sequencing, and gene-expression network analyses comparing normal aged brain to tissues from patients diagnosed with Late-onset Alzheimer's disease (LOAD) have identified protective and risk genes involved in microglial function and neuroinflammation. PLCG2 encodes the 1-phosphatidylinositol 4,5-bisphosphate phosphodiesterase gamma-2 (PLCγ2) enzyme, which converts phosphatidylinositol 4,5-bisphosphate [PI(4,5)P2] into 1D-myo-inositol 1,4,5-trisphosphate (IP3) and diacylglycerol (DAG) (FIG. 15A). PLCγ2 is mainly expressed in immune cells and microglia, but only sparsely expressed in other brain cells such as astrocytes and oligodendrocytes.¹ A rare coding variant of PLCG2, PLCG2P522R was identified using whole exome microarray analysis² and replicated in various populations.^{3,4,5} This variant was found to confer a protective effect. Alzheimer's patients who express this variant exhibit a slower rate of cognitive decline than patients who do not.² This variant has been characterized as a functional hypermorph.¹ Therefore, pharmacological intervention that increases the activity of wild-type PLCγ2 can provide a therapeutic strategy to reduce the rate of cognitive decline in Alzheimer's patients.

PLCγ2 is a complex multidomain protein that exists in an autoinhibited state with a regulatory domain blocking the enzyme's ability to bind to its substrate PIP2 at the cell membrane surface (FIGS. 15A-15C). Although the process is not fully understood, much can be inferred based on the known roles of Syk, BLNK, and Btk in B-cells^{6,7,8,9} and studies of PLCγ1 using hydrogen deuterium exchange (HDX).¹⁰ Within the TREM2 pathway, Syk is activated and phosphorylates BLNK allowing an activation complex of SYK, PLCγ2, and BTK (FIG. 16). This complex can then begin to separate PLCγ2's regulatory domain from the catalytic site, priming the enzyme for full activation. PLCγ2 is then phosphorylated at multiple sites by Syk and Btk with Tyr759 being the most crucial. Phosphorylated Tyr759 binds an arginine rich pocket within the cSH2 domain of PLCγ2, disrupting key interactions with the catalytic core and exposing membrane-binding surfaces and the active site. Molecular dynamics simulations have shown that the P522R protective variant causes a significant change to the position, structure, and flexibility of the nSH2 domain.¹¹ Thus, one strategy to activate PLCγ2 would be to identify molecules that bind at the SH2 domain interface and break interactions with the catalytic core domains.

Two molecules have been reported to activate phospholipase C (PLC) enzymes, U73122 (1-(6-((17β-3-methoxyestra-1,3,5 (10)-trien-17-yl)amino)hexyl)-1H-pyrrole-2,5-dione)¹² and m-3M3FBS (1) (2,4,6-trimethyl-N-(meta-3-trifluoromethyl-phenyl)-benzenesulfonamide).¹³ U73122 has been shown to activate PLCβ and PLCγ family members in vitro,¹² but it was originally identified as an inhibitor of PLCs.¹⁴ In fact, U73122 was extensively used as the prototypical inhibitor of PLC enzymes in cell culture.^{15,16,17,18,19} However, more recently U73122 has also been shown to affect numerous other cellular proteins including phospholipase D,²⁰ 5-lipoxygenase,²¹ calcium channels,²² potassium channels,²³ telomerase,²⁴ histamine H1 receptor,²⁵ and sarco/endoplasmic reticulum Ca2+-ATPase.²⁶ This complete lack of specificity is not surprising as the maleimide of U73122 can react with the thiol present in cysteine residues. Therefore, it is likely that U73122 reacts non-specifically with exposed cysteines on numerous proteins within a cell. This lack of specificity and high reactivity make U73122 a poor choice as a chemical probe to study the pharmacology of PLC activation and an unsuitable starting point for developing selective PLCγ2 activators.

m-3M3FBS was originally identified from a screen of compounds that enhanced superoxide-generating activity in human neutrophils.¹³ This activity was proposed to be caused by the direct activation of PLCs.¹³ Supporting this proposition, several labs have shown that one downstream signaling effect of PLC activation—intracellular calcium release—was observed in various cell types treated with m-3M3FBS.^{27,28,29,30,31} However, the potency and specificity of PLC activation of m-3M3FBS has been called into question, as there are reports that intracellular calcium release occurs before any PLC activity is detected, 32 and that m-3M3FBS effects the inward and outward flow of ions in a PLC-independent manner possibly through the direct inhibition of delayed rectifier potassium channels.³³ Additionally, m-3M3FBS has been shown to be cytotoxic in some cell lines.^{28,29,30} Thus, it has been unclear if m-3M3FBS exerts its activity primarily through PLC activation and if it does, whether analogs could be identified that are selective for PLCγ2. As no selective small molecule activators of PLCγ2 are currently known, there is a clear need to identify such an activator as pharmacological activation of PLCγ2 is a therapeutic strategy to treat Alzheimer's disease.

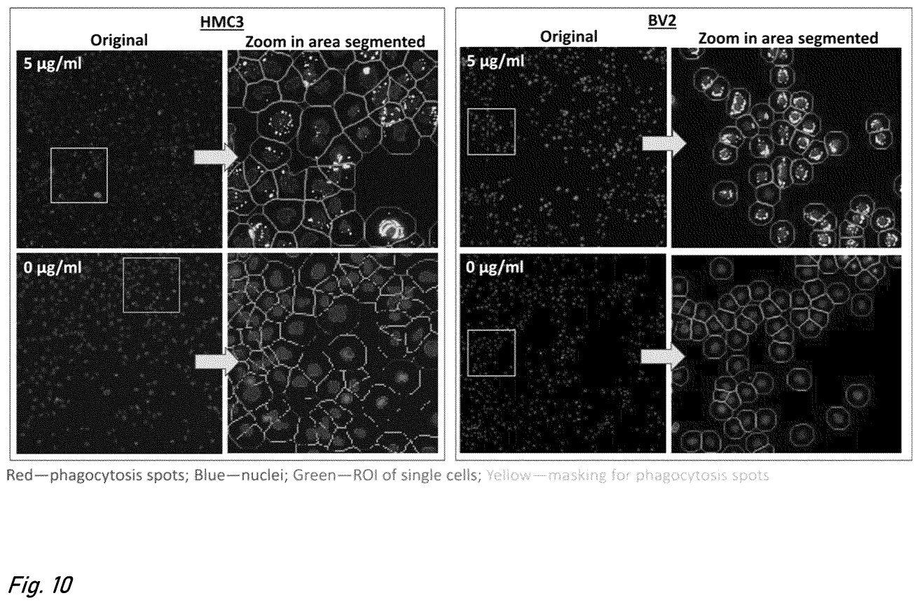

To assess its effectiveness and feasibility as a PLCγ2 activator, 1 (m-3M3FBS, TAD-0000040), the reported inactive analog 2 (0-3M3FBS, TAD-0000043), and a set of analogs (FIG. 28 and Table 4) were acquired via either synthesis or purchase. These compounds have been evaluated using a wide variety of assay classes namely target engagement utilizing cellular thermal shift assay (CETSA),³⁴ affinity selection mass spectrometry (ASMS),³⁵ biochemical activity employing the fluorogenic probe XY-69,³⁶ and cellular activity testing for phagocytosis. Cellular Thermal Shift Assays (CETSA) assess cellular target engagement by quantifying changes in protein thermal stability upon ligand binding to endogenous proteins in intact cells.³⁴ For SAR studies, a split Nano Luciferase (SplitLuc CETSA) version of the assay was utilized to provide sufficient throughput.^{37,38} HMC3 cells were stably transfected with PLCγ2-HiBit to enable protein expression in a physiologically relevant cellular context. The thermal stability of this HiBit-labeled protein in intact cells was measured in the following two formats. In the initial screen, using 96-well plates, cells were treated with FBS analogs at a single concentration (100 μM) at 37° C. for 60 min and then exposed to a 3-minute isothermal heating step at 45.9° C. (experimentally determined Tm of PLCγ2-HiBit). Luminescence detection was then conducted with the Nano-Glo HiBit Lytic detection kit (Promega, Cat. N3040).³⁸ Compound treatment was performed in triplicate, and raw signals were normalized to the mean of the controls (in the same plate) as % of control. When the difference in compound-treated cells >control mean+3SD, the compound was considered positive and selected for further testing with dose-response CETSA. In the dose response CETSA, cells were treated with decreasing concentrations from 100 μM with 1:3 serial dilutions to generate an 8-point curve. An AC₅₀ was calculated with the % of control data using a four-parameter logistic curve regression model, and the percent luminescence remaining at the highest concentration is also reported for the compounds when the difference from the DMSO control is greater than 3× the standard deviation (SD). Otherwise, an AC₅₀ was not calculated. This dose response CETSA data (FIG. 10 and Table 5) indicate that several analogs demonstrated superior target engagement compared to 1. However, numerous analogs exhibited a similar response in the absence of the PLCγ2 enzyme, indicating promiscuity of these compounds or the possibility of a false positive result. As an example TAD-0058715 showed evidence of target engagement (54%) without a significant loss of signal in the HiBit only control (95%).

TABLE 4
Arylsulfonamide literature compounds and selected analogs

| | | | | | | | | | | CNS |
|---|---|---|---|---|---|---|---|---|---|---|
| | | | MW | | | | H-bond | H-bond | Lipinski | MPO |
| Compound | Name | TADⁱ | (g/mol) | cLogPⁱⁱ | cLogDⁱⁱⁱ | tPSA^{iv} | donors | acceptors | violations^{v} | score^{vi} |
| 1 | m-3M3FBS | TAD-0000040 | 343.4 | 4.88 | 4.81 | 46.17 | 1 | 3 | 0 | 3.89 |
| 2 | o-3M3FBS | TAD-0000043 | 343.4 | 4.88 | 4.68 | 46.17 | 1 | 3 | 0 | 3.89 |
| 3 | — | TAD-0058693 | 300.4 | 3.86 | 3.79 | 69.96 | 1 | 4 | 0 | 4.51 |
| 4 | — | TAD-0058698 | 301.3 | 3.34 | 3.21 | 46.17 | 1 | 3 | 0 | 5.06 |
| 5 | — | TAD-0058715 | 289.4 | 4.53 | 3.41 | 46.17 | 1 | 3 | 0 | 4.36 |
| 6 | — | TAD-0058717 | 329.3 | 4.37 | 4.29 | 46.17 | 1 | 3 | 0 | 4.15 |
| 7 | — | TAD-0058725 | 325.4 | 4.39 | 4.33 | 46.17 | 1 | 3 | 0 | 4.14 |
| 8 | — | TAD-0410954 | 336.2 | 3.32 | 3.17 | 46.17 | 1 | 3 | 0 | 5.09 |
| 9 | — | TAD-0410993 | 370.2 | 4.55 | 4.16 | 46.17 | 1 | 3 | 0 | 3.99 |
| 10 | — | TAD-0411016 | 394.2 | 4.62 | 4.51 | 46.17 | 1 | 3 | 0 | 3.78 |
| 11 | — | TAD-0411056 | 282.4 | 3.02 | 0.79 | 58.20 | 2 | 4 | 0 | 4.49 |

ⁱTREAT-AD serial number.
ⁱⁱChemAxon calculated partition coefficient of the ratio of the concentration of the compound in octanol to its concentration in water.
ⁱⁱⁱChemAxon calculated octanol-water distribution coefficient (from pKa and cLogP);
^{iv}Topological polar surface area (Å2) based on the method of Ertl et al.;³⁹
^{v}See Lipinski et al.;⁴⁰
^{vi}Central Nervous System Multiparameter Optimization score based on the method of Wager et al.⁴¹

TABLE 5
Cellular thermal shift assay results

| | | AC50 | PLCγ2-HiBit | HiBit |
|---|---|---|---|---|
| Compound | TAD | (μM)ⁱ | %ⁱⁱ | only %ⁱⁱⁱ |
| 1 | TAD-0000040 | NC | 105 | N.T. |
| 2 | TAD-0000043 | NC | 102 | N.T. |
| 3 | TAD-0058693 | NC | 96 | N.T. |
| 4 | TAD-0058698 | NC | 65 | 82 |
| 5 | TAD-0058715 | >100 | 54 | 95 |
| 6 | TAD-0058717 | 99 | 51 | 74 |
| 7 | TAD-0058725 | NC | 78 | 104 |
| 8 | TAD-0410954 | >100 | 66 | 44 |
| 9 | TAD-0410993 | 71 | 32 | 38 |
| 10 | TAD-0411016 | 57 | 17 | 40 |
| 11 | TAD-0411056 | NC | 64 | 93 |

ⁱConcentration (μM) that induced a half-maximum loss of luminesce (AC₅₀) compared to control.
ⁱⁱPercent luminescence at highest dose (100 μM) compared to control (DMSO).
ⁱⁱⁱPercent luminescence at highest dose (100 μM) with HiBit only compared to control (DMSO).

As an orthogonal approach for investigating the target engagement of this series, affinity selection mass spectrometry (ASMS) was employed to test a subset of compounds. Using a dose range of 100-0.05 μM in 384 well low-volume polypropylene microtiter plates, a solution of PLCγ2 (final concentration 400 nM) was transferred to biochip arrays functionalized with a Tris-Ni-NTA-presenting self-assembled monolayer to immobilize the His-tagged protein. The arrays were incubated for 90 min in a humidified chamber to prevent evaporation and allow for specific immobilization of the His-PLCγ2 protein, along with any bound small molecules. The arrays were purified by a rapid <3 second gentle wash step with deionized ultra-filtered water and dried with compressed air. ASMS was performed using the reflector positive mode on an Aβ Sciex TOF-TOF 5800 System (Aβ Sciex, Framingham, MA). While ATP showed clear separation from background signal, neither 1 or 2 indicated substantial evidence of target engagement (FIG. 18A-18C).

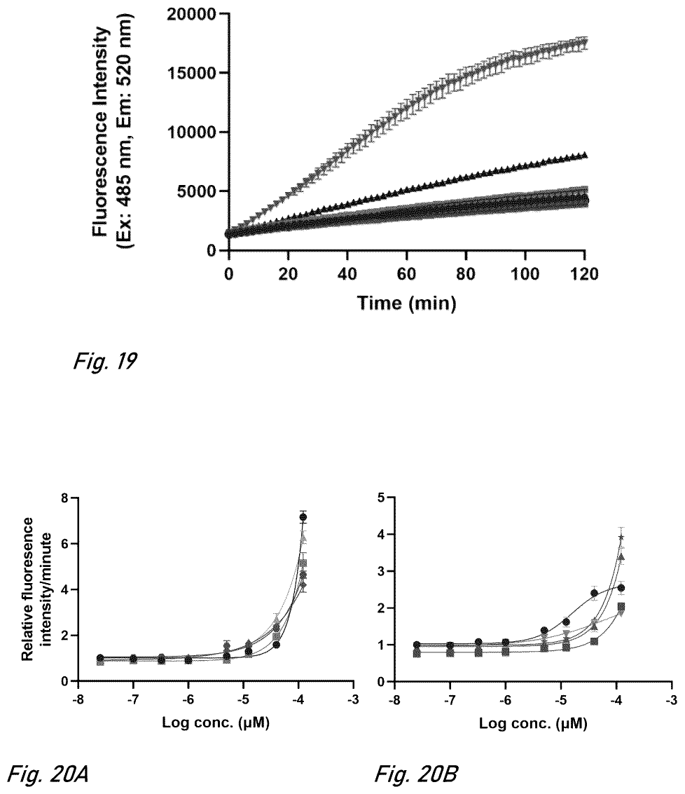

Sondek and coworkers have described an assay for PLCγ1 activity in the presence of liposomes as a model for activity at the cell membrane where the natural ligand is located.³⁶ The assay was redeveloped and reoptimized for PLCγ2 at the Purdue Institute for Drug Discovery. Liposomes containing XY-69 (purchased from Avanti Polar Lipids, cat. #850168) were generated using phosphatidylinositol 4,5-bisphosphate (PIP2) and 1-stearoyl-2-arachidonoyl-sn-glycero-3-phosphatidylethanolamine (lipidPE). The PLCγ2 enzyme was exposed to this solution and, after treatment with the compounds of interest, the fluorescence intensity (ex: 485 nm; em: 520 nm) was recorded every 2 min for 120 min (FIG. 19). The slope of the linear portion of the curves (up to 50 min) was compared to control giving a relative rate of reaction. Dose dependent curves of a representative sample of the compounds tested are shown (FIG. 20A-20B).

Both aromatic rings tolerate a wide range of substitutions; however, in nearly all instances, this activity was lost when the rings were replaced with aliphatic functionality. For example, TAD-0411056 showed no enzymatic activation in the single dose at 100 μM, thus it was not submitted for dose response testing. Several compounds containing alterations to the sulfonamide linker (reverse sulfonamide, sulfonate, sulfide, and amine) retained activity while others (amide, sulfone, and sulfoxide) did not. The reported PLC activator 1 (TAD-0000040) gave among the highest increases of all analogs tested. As expected, the change in slope of the reported inactive analog 2 (TAD-0000043) was much lower, though it still showed a statistically significant increase in activation.

A phenotypic high-content imaging assay with simultaneous measurements of phagocytosis, cell number, and nuclear intensity was used to assess both cellular pharmacology and cell health. Model cell lines BV2 (immortalized mouse microglia, a gift from Dr. Michelle Block's laboratory at IUSM) and HMC3 (immortalized human microglia, obtained from ATCC, CRL-3304) were used to provide adequate throughput. Cells were cultured in DMEM GlutaMax media (ThermoFisher) containing 10% FBS and Pen-Strep in a 37° C. 5% CO₂ incubator, plated in Corning Falcon 384 well Optilux Black and clear bottom plates: 400 cells/45 μl/well (BV2 cells), 600 cells/45 μl/well (HMC3 cells) and 2000 cell/45 μl/well (primary cells), and treated with 10× serially diluted compounds (60 μM-3 nM) for 48 hrs at 37° C. The cells were then seeded with pHrodo™-myelin (20 hrs total) 24 hrs after compound treatment initiation; 1 mg/ml (protein equivalent) pHrodo™-myelin stocks were stored at −20° C. or −80° C., thawed, diluted with culture media to produce a 10× seeding solution (50 μg/ml) and added to the 384-well plate (5 μl/well). Nuclear staining solution (1 μl of 10 mg/ml Hoechst-33342 per 1 ml culture media; 20 μl/well) was added to a final concentration of 2.5 μg/ml, and the plates were incubated for 30 min at 37° C. and then scanned with an ArrayScan™ automatic high content imaging system (10× objective lens, 4 fields/well collected). Mean total phagocytosis spot intensity per cell, total cell counts per well, and mean average nuclear intensity per cell for cell health were measured. Cellular potencies for each endpoint (EC₅₀ for phagocytosis, IC₅₀ for cell count) were calculated using a four-parameter logistic curve regression model.

TABLE 6
Phagocytosis results

| | | Ave. % Phagocytosis | | Ave. % Cell Count | | |
|---|---|---|---|---|---|---|
| | | @ 60 uMⁱ | | @ 60 uMⁱ | | |
| Com- | | HMC3 | BV2 | HMC3 | BV2 | |
| pound | Name | cells | cells | cells | cells | N |
| 1 | m-3M3FBS | 0 | 0.05 | 3 | 2 | 2 |
| 2 | o-3M3FBS | 99 | 104 | 103 | 104 | 2 |
| 3 | TAD-0058693 | 111 | 113 | 79 | 85 | 2 |
| 4 | TAD-0058698 | 89 | 106 | 84 | 95 | 2 |
| 5 | TAD-0058715 | 111 | 165 | 78 | 45 | 1 |
| 6 | TAD-0058717 | 86 | 118 | 62 | 85 | 2 |
| 7 | TAD-0058725 | 0 | 0 | 6 | 2 | 2 |
| 8 | TAD-0410954 | 104 | 114 | 88 | 86 | 2 |
| 9 | TAD-0410993 | 281 | 581 | 16 | 9 | 1 |
| 10 | TAD-0411016 | 93 | 434 | 11 | 1 | 1 |
| 11 | TAD-0411056 | 146 | 225 | 65 | 63 | 1 |

ⁱPercent luminescence at highest dose (60 μM) compared to control.

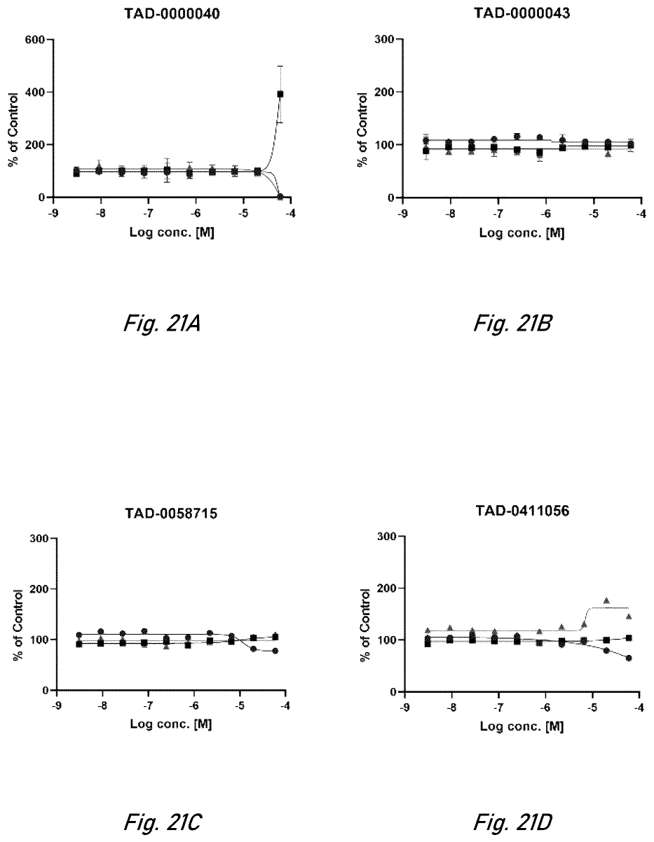

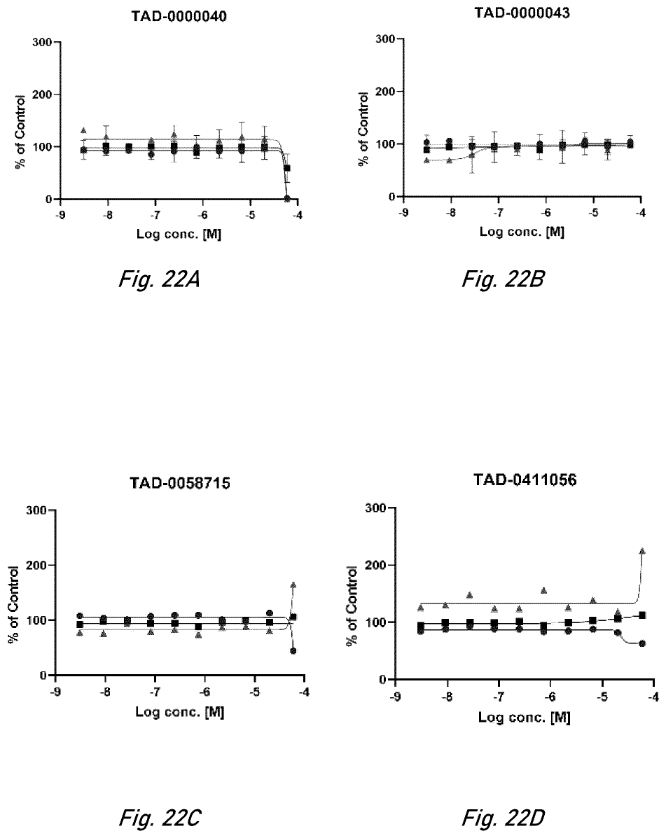

Ideally a PLCγ2 activator would induce an increase in phagocytosis at concentrations lower than the onset of a decrease in the cell count. Any apparent increase in cell activity that is accompanied by a sharp decline in the cell count confound interpretation. Many of the FBS analogs showed no significant increase in phagocytosis, or in the case of 5 (TAD-0058715), 9 (TAD-0410993), and 10 (TAD-0411016) the increase was paired with a sharp cell count decline (Table 6 and FIGS. 21A-21D, 22A-22D). The exception being 11 (TAD-0411056) which still suffers from a moderate degree of potential cytotoxicity.

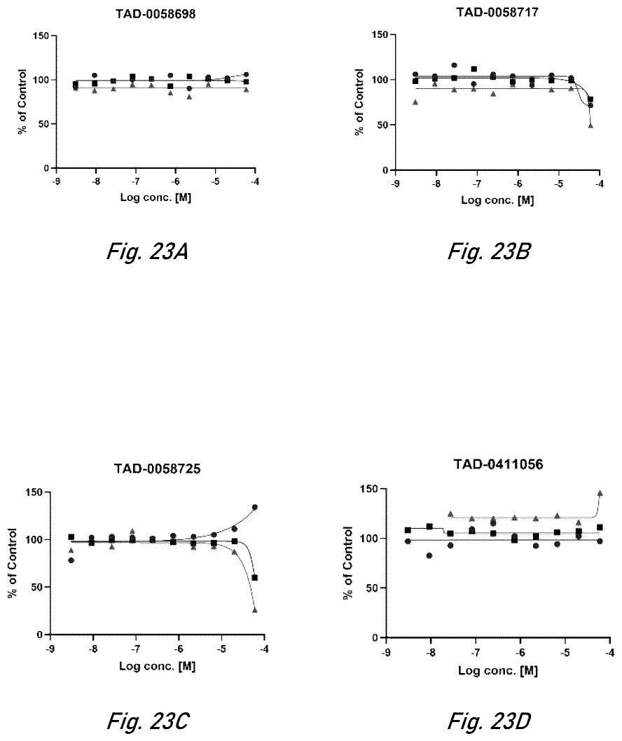

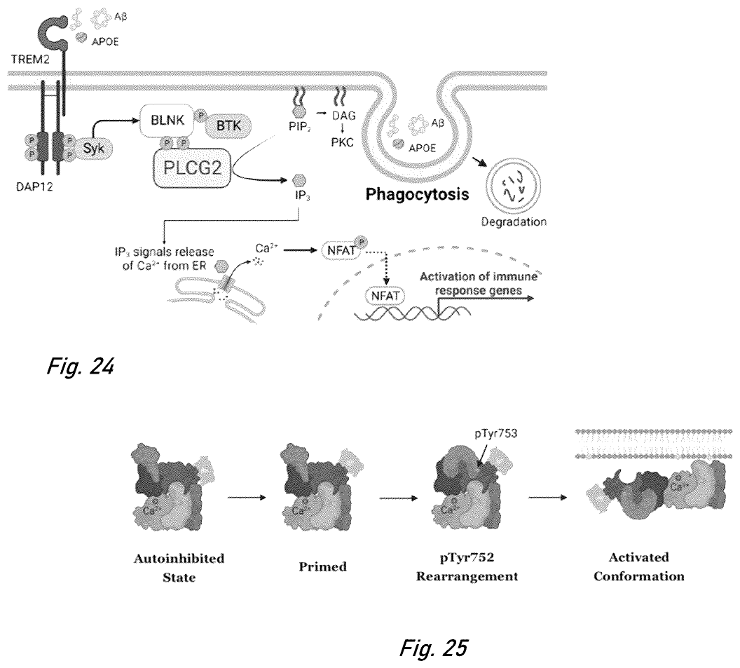

Primary mouse microglia isolated from mouse brain were periodically tested in the phagocytosis assay to verify that readouts from the BV2 and HMC3 cells are mechanistically similar to those obtained with primary cells. Primary microglia were obtained from the cortical tissue of B6J neonatal mice (P0-P3), which was homogenized in DMEM, filtered through 250 and 100 μm mesh, and cultured in Advanced DMEM/F12+10% FBS, 1× GlutaMAX and 1× Pen/Strep. At 21 days in vitro (DIV), the cultures were subjected to mild trypsinization using 0.083% Trypsin-EDTA in DMEM for 30 min to detach an intact layer of astrocytes. A select number of cell active FBS analogs were tested in these cells, but unfortunately, little to no increase in phagocytosis was manifested (FIGS. 23A-23D).

Physicochemical and ADME properties were determined using the following assays: microsomal stability intrinsic clearance (Clint) in mouse liver microsomal solution, MDCK permeability (Papp), protein binding (fu) in mouse plasma, and kinetic solubility in pH 7.4 phosphate buffer (Table 7). Many of these analogs performed poorly in these assays, particularly in the microsomal stability intrinsic clearance assay where many failed to give any meaningful response.

TABLE 7
Measured physicochemical and ADME properties.

| | | | | | Kinetic |
|---|---|---|---|---|---|
| Com- | | Clᵢₙₜ | Papp | | solubility |
| pound | Name | μL/min/mg)ⁱ | (×10−⁶ cm/s)ⁱⁱ | fᵤⁱⁱⁱ | (μM)^{iv} |
| 1 | TAD-0000040 | * | * | NT | 21-40 |
| 3 | TAD-0058693 | * | 69.8 | 0.0003 | 81-100 |
| 4 | TAD-0058698 | 607.66 | 79.7 | NT | >100 |
| 6 | TAD-0058717 | * | 17.1 | 0.003 | 61-80 |
| 7 | TAD-0058725 | * | 37.4 | NT | 41-60 |
| 8 | TAD-0410954 | 77.83 | 95.0 | 0.0193 | >100 |

* = Assays failed when tested.
NT = not tested.
ⁱIntrinsic clearance in mouse liver microsomes.
ⁱⁱMDCK Permeability assay as an estimate of intestinal absorption.
iiiMouse plasma fraction unbound.
^{iv}Kinetic solubility in pH 7.4 phosphate buffer.

To investigate the biological function of PLCγ2 and study the pharmacology of PLCγ2 activation, rigorously validated molecular tools are essential⁴². Therefore, we have developed assays and characterized reported activators to further advance research in this area. Although many showed activation in the biochemical XY-69 assay, the majority of the FBS analogs tested failed to show significant levels of target engagement in CETSA or increase in phagocytosis in primary mouse microglia cells. These results coupled with the poor performance in ADME assays indicate that 1 is a poor starting point for a PLCγ2 drug discovery program. We have screened for novel activators, characterized them in the assays described here and are evaluating the systemic and central exposure of these compounds in mice.

### REFERENCES

### References for Example 2

Andreone B J, Przybyla L, Llapashtica C, Rana A, Davis S S, van Lengerich B, . . . . Lewcock J W (2020). Alzheimer's-associated PLCgamma2 is a signaling node required for both TREM2 function and the inflammatory response in human microglia. Nat Neurosci, 23 (8), 927-938. doi: 10.1038/s41593-020-0650-6
Blasi E, Barluzzi R, Bocchini V, Mazzolla R, & Bistoni F (1990). Immortalization of murine microglial cells by a v-raf/v-myc carrying retrovirus. J Neuroimmunol, 27 (2-3), 229-237. doi: 10.1016/0165-5728 (90) 90073-v
Casella J F, Flanagan M D, & Lin S (1981). Cytochalasin D inhibits actin polymerization and induces depolymerization of actin filaments formed during platelet shape change. Nature, 293 (5830), 302-305. doi: 10.1038/293302a0
Cunningham C L, Martinez-Cerdeno V, & Noctor S C (2013). Microglia Regulate the Number of Neural Precursor Cells in the Developing Cerebral Cortex. Journal of Neuroscience, 33 (10), 4216-4233. doi: 10.1523/Jneurosci.3441-12.2013
Desale S E, & Chinnathambi S (2021). Phosphoinositides signaling modulates microglial actin remodeling and phagocytosis in Alzheimer's disease. Cell Commun Signal, 19 (1), 28. doi: 10.1186/s12964-021-00715-0
Dhawan G, & Combs C K (2012). Inhibition of Src kinase activity attenuates amyloid associated microgliosis in a murine model of Alzheimer's disease. J Neuroinflammation, 9, 117. doi: 10.1186/1742-2094-9-117
Edler M K, Mhatre-Winters I, & Richardson J R (2021). Microglia in Aging and Alzheimer's Disease: A Comparative Species Review. Cells, 10 (5). doi: 10.3390/cells10051138
Ennerfelt H, Frost E L, Shapiro D A, Holliday C, Zengeler K E, Voithofer G, . . . . Lukens J R (2022). SYK coordinates neuroprotective microglial responses in neurodegenerative disease. Cell, 185 (22), 4135-4152 e4122. doi: 10.1016/j.cell.2022.09.030
Erwig M S, Hesse D, Jung R B, Uecker M, Kusch K, Tenzer S, . . . . Werner H B (2019). Myelin: Methods for Purification and Proteome Analysis. Oligodendrocytes, 1936, 37-63. doi: 10.1007/978-1-4939-9072-6_3
Franco-Bocanegra D K, Gourari Y, McAuley C, Chatelet D S, Johnston D A, Nicoll J A R, & Boche D (2021). Microglial morphology in Alzheimer's disease and after Abeta immunotherapy. Sci Rep, 11 (1), 15955. doi: 10.1038/s41598-021-95535-0
Gillooly D J, Simonsen A, & Stenmark H (2001). Phosphoinositides and phagocytosis. J Cell Biol, 155 (1), 15-17. doi: 10.1083/jcb.200109001
Gyoneva S, Swanger S A, Zhang J, Weinshenker D, & Traynelis S F (2016). Altered motility of plaque-associated microglia in a model of Alzheimer's disease. Neuroscience, 330, 410-420. doi: 10.1016/j.neuroscience.2016.05.061
Harvath L, & Terle D A (1999). Assay for phagocytosis. Methods Mol Biol, 115, 281-290. doi: 10.1385/1-59259-213-9:281 Janabi N, Peudenier S, Heron B, Ng K H, & Tardieu M (1995). Establishment of human microglial cell lines after transfection of primary cultures of embryonic microglial cells with the SV40 large T antigen. Neurosci Lett, 195 (2), 105-108. doi: 10.1016/0304-3940 (94) 11792-h
Kettenmann H, Hanisch U K, Noda M, & Verkhratsky A (2011). Physiology of microglia. Physiol Rev, 91 (2), 461-553. doi: 10.1152/physrev.00011.2010
Lewcock J W, Schlepckow K, Di Paolo G, Tahirovic S, Monroe K M, & Haass C (2020). Emerging Microglia Biology Defines Novel Therapeutic Approaches for Alzheimer's Disease. Neuron, 108 (5), 801-821. doi: 10.1016/j.neuron.2020.09.029
Li Q Y, & Barres B A (2018). Microglia and macrophages in brain homeostasis and disease. Nature Reviews Immunology, 18 (4), 225-242. doi: 10.1038/nri.2017.125 Margadant C, Cremers L, Sonnenberg A, & Boonstra J (2013). MAPK uncouples cell cycle progression from cell spreading and cytoskeletal organization in cycling cells. Cell Mol Life Sci, 70 (2), 293-307. doi: 10.1007/s00018-012-1130-2
Matcovitch-Natan O, Winter D R, Giladi A, Aguilar S V, Spinrad A, Sarrazin S, . . . . Amit I (2016). Microglia development follows a stepwise program to regulate brain homeostasis. Science, 353 (6301). doi: ARTN aad8670 10.1126/science.aad8670
Meena J, Goswami D G, Anish C, & Panda A K (2021). Cellular uptake of polylactide particles induces size dependent cytoskeletal remodeling in antigen presenting cells. Biomater Sci, 9 (23), 7962-7976. doi: 10.1039/dlbm01312b
Melzer C, von der Ohe J, & Hass R (2019). Involvement of Actin Cytoskeletal Components in Breast Cancer Cell Fusion with Human Mesenchymal Stroma/Stem-Like Cells. Int J Mol Sci, 20 (4). doi: 10.3390/ijms20040876
Mundt S, Greter M, & Becher B (2022). The CNS mononuclear phagocyte system in health and disease. Neuron, 110 (21), 3497-3512. doi: 10.1016/j.neuron.2022.10.005
Murai M, Seki K, Sakurada J, Usui A, & Masuda S (1993). Effects of cytochalasins B and D on Staphylococcus aureus adherence to and ingestion by mouse renal cells from primary culture. Microbiol Immunol, 37 (1), 69-73. doi: 10.1111/j.1348-0421.1993.tb03181.x
Nimmerjahn A, Kirchhoff F, & Helmchen F (2005). Resting microglial cells are highly dynamic surveillants of brain parenchyma in vivo. Science, 308 (5726), 1314-1318. doi: 10.1126/science.1110647
Nizami S, Hall-Roberts H, Warrier S, Cowley S A, & Di Daniel E (2019). Microglial inflammation and phagocytosis in Alzheimer's disease: Potential therapeutic targets. Br J Pharmacol, 176 (18), 3515-3532. doi: 10.1111/bph.14618
Nugent A A, Lin K, van Lengerich B, Lianoglou S, Przybyla L, Davis S S, . . . . Di Paolo G (2020). TREM2 Regulates Microglial Cholesterol Metabolism upon Chronic Phagocytic Challenge. Neuron, 105 (5), 837-854 e839. doi: 10.1016/j.neuron.2019.12.007
Nygaard H B (2018). Targeting Fyn Kinase in Alzheimer's Disease. Biol Psychiatry, 83 (4), 369-376. doi: 10.1016/j.biopsych.2017.06.004
Nygaard H B, Wagner A F, Bowen G S, Good S P, MacAvoy M G, Strittmatter K A, . . . van Dyck C H (2015). A phase Ib multiple ascending dose study of the safety, tolerability, and central nervous system availability of AZD0530 (saracatinib) in Alzheimer's disease. Alzheimers Res Ther, 7 (1), 35. doi: 10.1186/s13195-015-0119-0
Phanse Y, Ramer-Tait A E, Friend S L, Carrillo-Conde B, Lueth P, Oster C J, . . . . Bellaire B H (2012). Analyzing cellular internalization of nanoparticles and bacteria by multi-spectral imaging flow cytometry. J Vis Exp (64), e3884. doi: 10.3791/3884
Plescher M, Seifert G, Hansen J N, Bedner P, Steinhauser C, & Halle A (2018). Plaque-dependent morphological and electrophysiological heterogeneity of microglia in an Alzheimer's disease mouse model. Glia, 66 (7), 1464-1480. doi: 10.1002/glia.23318
Potjewyd F M, Annor-Gyamfi J K, Aube J, Chu S, Conlon I L, Frankowski K J, . . . . Axtman A D (2022a). Use of AD Informer Set compounds to explore validity of novel targets in Alzheimer's disease pathology. Alzheimers Dement (N Y), 8 (1), e12253. doi: 10.1002/trc2.12253
Potjewyd F M, Annor-Gyamfi J K, Aube J, Chu S, Conlon I L, Frankowski K J, . . . . Axtman A D (2022b). AD Informer Set: Chemical tools to facilitate Alzheimer's disease drug discovery. Alzheimers Dement (N Y), 8 (1), e12246. doi: 10.1002/trc2.12246
Salter M W, & Stevens B (2017). Microglia emerge as central players in brain disease. Nature Medicine, 23 (9), 1018-1027. doi: 10.1038/nm.4397
Saura J, Tusell J M, & Serratosa J (2003). High-yield isolation of murine microglia by mild trypsinization. Glia, 44 (3), 183-189. doi: 10.1002/glia.10274
Somoza J R, Koditek D, Villasenor A G, Novikov N, Wong M H, Liclican A, . . . . McGrath M E (2015). Structural, biochemical, and biophysical characterization of idelalisib binding to phosphoinositide 3-kinase delta. J Biol Chem, 290 (13), 8439-8446. doi: 10.1074/jbc.M114.634683
Streit W J, Mrak R E, & Griffin W S (2004). Microglia and neuroinflammation: a pathological perspective. J Neuroinflammation, 1 (1), 14. doi: 10.1186/1742-2094-1-14
Taetzsch T, Levesque S, McGraw C, Brookins S, Luqa R, Bonini M G, . . . . Block M L (2015). Redox regulation of NF-kappaB p50 and MI polarization in microglia. Glia, 63 (3), 423-440. doi: 10.1002/glia.22762
Tilney L G, & Portnoy D A (1989). Actin filaments and the growth, movement, and spread of the intracellular bacterial parasite, Listeria monocytogenes. J Cell Biol, 109 (4 Pt 1), 1597-1608. doi: 10.1083/jcb. 109.4.1597
van Dyck C H, Nygaard H B, Chen K, Donohue M C, Raman R, Rissman R A, . . . . Strittmatter S M (2019). Effect of AZD0530 on Cerebral Metabolic Decline in Alzheimer Disease: A Randomized Clinical Trial. JAMA Neurol, 76 (10), 1219-1229. doi: 10.1001/jamaneurol.2019.2050
Vance D T, Dufresne J, Florentinus-Mefailoski A, Tucholska M, Trimble W, Grinstein S, & Marshall J G (2016). A phagocytosis assay for oxidized low-density lipoprotein versus immunoglobulin G-coated microbeads in human U937 macrophages. Anal Biochem, 500, 24-34. doi: 10.1016/j.ab.2016.01.007
Wake H, Moorhouse A J, Miyamoto A, & Nabekura J (2013). Microglia: actively surveying and shaping neuronal circuit structure and function. Trends Neurosci, 36 (4), 209-217. doi: 10.1016/j.tins.2012.11.007
Witting A, Muller P, Herrmann A, Kettenmann H, & Nolte C (2000). Phagocytic clearance of apoptotic neurons by Microglia/Brain macrophages in vitro: involvement of lectin-, integrin-, and phosphatidylserine-mediated recognition. J Neurochem, 75 (3), 1060-1070. doi: 10.1046/j.1471-4159.2000.0751060.x
Yeo J C, Wall A A, Stow J L, & Hamilton N A (2013). High-throughput quantification of early stages of phagocytosis. Biotechniques, 55 (3), 115-124. doi: 10.2144/000114075

### References for Example 3

ADDIN EN.REFLIST 1 Magno, L. et al. Alzheimer's disease phospholipase C-gamma-2 (PLCG2) protective variant is a functional hypermorph. Alzheimers Res Ther 11, 16 (2019). https://doi.org: 10.1186/s13195-019-0469-0
2 Sims, R. et al. Rare coding variants in PLCG2, ABI3, and TREM2 implicate microglial-mediated innate immunity in Alzheimer's disease. Nat Genet 49, 1373-1384 (2017). https://doi.org: 10.1038/ng.3916
3 Conway, O. J. et al. ABI3 and PLCG2 missense variants as risk factors for neurodegenerative diseases in Caucasians and African Americans. Mol Neurodegener 13, 53 (2018). https://doi.org: 10.1186/s13024-018-0289-x
4 van der Lee, S. J. et al. A nonsynonymous mutation in PLCG2 reduces the risk of Alzheimer's disease, dementia with Lewy bodies and frontotemporal dementia, and increases the likelihood of longevity. Acta Neuropathol 138, 237-250 (2019). https://doi.org: 10.1007/s00401-019-02026-8
5 Dalmasso, M. C. et al. Transethnic meta-analysis of rare coding variants in PLCG2, ABI3, and TREM2 supports their general contribution to Alzheimer's disease. Transl Psychiatry 9, 55 (2019). https://doi.org: 10.1038/s41398-019-0394-9
6 Fu, C., Turck, C. W., Kurosaki, T. & Chan, A. C. BLNK: a central linker protein in B cell activation. Immunity 9, 93-103 (1998). https://doi.org: 10.1016/s1074-7613 (00) 80591-9
7 Hashimoto, S. et al. Identification of the SH2 domain binding protein of Bruton's tyrosine kinase as BLNK—functional significance of Btk-SH2 domain in B-cell antigen receptor-coupled calcium signaling. Blood 94, 2357-2364 (1999).
8 Ishiai, M., Sugawara, H., Kurosaki, M. & Kurosaki, T. Cutting edge: association of phospholipase C-gamma 2 Src homology 2 domains with BLNK is critical for B cell antigen receptor signaling. J Immunol 163, 1746-1749 (1999).
9 Watanabe, D. et al. Four tyrosine residues in phospholipase C-gamma 2, identified as Btk-dependent phosphorylation sites, are required for B cell antigen receptor-coupled calcium signaling. J Biol Chem 276, 38595-38601 (2001). https://doi.org: 10.1074/jbc.M103675200
10 Siraliev-Perez, E. et al. Dynamics of allosteric regulation of the phospholipase C-gamma isozymes upon recruitment to membranes. Elife 11 (2022). https://doi.org: 10.7554/eLife.77809
11 Maguire, E. et al. PIP2 depletion and altered endocytosis caused by expression of Alzheimer's disease-protective variant PLCgamma2 R522. EMBO J 40, e105603 (2021). https://doi.org: 10.15252/embj.2020105603
12 Klein, R. R. et al. Direct activation of human phospholipase C by its well known inhibitor u73122. J Biol Chem 286, 12407-12416 (2011). https://doi.org: 10.1074/jbc.M110.191783
13 Bae, Y. S. et al. Identification of a compound that directly stimulates phospholipase C activity. Mol Pharmacol 63, 1043-1050 (2003). https://doi.org: 10.1124/mol.63.5.1043
14 Bleasdale, J. E. et al. Inhibition of phospholipase C dependent processes by U-73, 122. Adv Prostaglandin Thromboxane Leukot Res 19, 590-593 (1989).
15 Carvou, N., Norden, A. G., Unwin, R. J. & Cockcroft, S. Signalling through phospholipase C interferes with clathrin-mediated endocytosis. Cell Signal 19, 42-51 (2007). https://doi.org: 10.1016/j.cellsig.2006.05.023
16 Hou, C. et al. In vivo activity of a phospholipase C inhibitor, 1-(6-((17beta-3-methoxyestra-1,3,5 (10)-trien-17-yl)amino)hexyl)-1H-pyrrole-2,5-di one (U73122), in acute and chronic inflammatory reactions. J Pharmacol Exp Ther 309, 697-704 (2004). https://doi.org: 10.1124/jpet. 103.060574
17 Smith, R. J. et al. Receptor-coupled signal transduction in human polymorphonuclear neutrophils: effects of a novel inhibitor of phospholipase C-dependent processes on cell responsiveness. J Pharmacol Exp Ther 253, 688-697 (1990).
18 Thompson, A. K., Mostafapour, S. P., Denlinger, L. C., Bleasdale, J. E. & Fisher, S. K. The aminosteroid U-73122 inhibits muscarinic receptor sequestration and phosphoinositide hydrolysis in SK—N-SH neuroblastoma cells. A role for Gp in receptor compartmentation. J Biol Chem 266, 23856-23862 (1991).
19 Ward, P. D., Ouyang, H. & Thakker, D. R. Role of phospholipase C-beta in the modulation of epithelial tight junction permeability. J Pharmacol Exp Ther 304, 689-698 (2003). https://doi.org: 10.1124/jpet. 102.043638
20 Burgdorf, C., Schafer, U., Richardt, G. & Kurz, T. U73122, an aminosteroid phospholipase C inhibitor, is a potent inhibitor of cardiac phospholipase D by a PIP2-dependent mechanism. J Cardiovasc Pharmacol 55, 555-559 (2010). https://doi.org: 10.1097/FJC.0b013e3181d8bec5
21 Feisst, C., Albert, D., Steinhilber, D. & Werz, O. The aminosteroid phospholipase C antagonist U-73122 (1-[6-[[17-beta-3-methoxyestra-1,3,5 (10)-trien-17-yl]amino]hexyl]-1H-pyrrole-2,5-dione) potently inhibits human 5-lipoxygenase in vivo and in vitro. Mol Pharmacol 67, 1751-1757 (2005). https://doi.org: 10.1124/mol. 105.011007
22 Pulcinelli, F. M., Gresele, P., Bonuglia, M. & Gazzaniga, P. P. Evidence for separate effects of U73122 on phospholipase C and calcium channels in human platelets. Biochem Pharmacol 56, 1481-1484 (1998). https://doi.org: 10.1016/s0006-2952 (98) 00146-4
23 Klose, A., Huth, T. & Alzheimer, C. 1-[6-[[(17beta)-3-methoxyestra-1,3,5 (10)-trien-17-yl]amino]hexyl]-1H-pyrrole-2,5-dione (U73122) selectively inhibits Kir3 and BK channels in a phospholipase C-independent fashion. Mol Pharmacol 74, 1203-1214 (2008). https://doi.org: 10.1124/mol.108.047837
24 Chen, Y. J., Sheng, W. Y., Huang, P. R. & Wang, T. C. Potent inhibition of human telomerase by U-73122. J Biomed Sci 13, 667-674 (2006). https://doi.org: 10.1007/s11373-006-9100-z
25 Hughes, S. A., Gibson, W. J. & Young, J. M. The interaction of U-73122 with the histamine H1 receptor: implications for the use of U-73122 in defining H1 receptor-coupled signalling pathways. Naunyn Schmiedebergs Arch Pharmacol 362, 555-558 (2000). https://doi.org: 10.1007/s002100000326
26 Macmillan, D. & McCarron, J. G. The phospholipase C inhibitor U-73122 inhibits Ca (2+) release from the intracellular sarcoplasmic reticulum Ca (2+) store by inhibiting Ca (2+) pumps in smooth muscle. Br J Pharmacol 160, 1295-1301 (2010). https://doi.org: 10.1111/j.1476-5381.2010.00771.x
27 Szebenyi, S. A. et al. Increases in intracellular calcium via activation of potentially multiple phospholipase C isozymes in mouse olfactory neurons. Front Cell Neurosci 8, 336 (2014). https://doi.org: 10.3389/fncel.2014.00336
28 Jung, E. M. et al. The novel phospholipase C activator, m-3M3FBS, induces apoptosis in tumor cells through caspase activation, down-regulation of XIAP and intracellular calcium signaling. Apoptosis 13, 133-145 (2008). https://doi.org: 10.1007/s10495-007-0159-4
29 Liu, S. I. et al. M-3M3FBS-induced Ca (2) (+) movement and apoptosis in HA59T human hepatoma cells. Chin J Physiol 56, 26-35 (2013). https://doi.org: 10.4077/CJP.2013.BAA091
30 Chen, W. C. et al. Rise of [Ca (2) (+)]i and apoptosis induced by M-3M3FBS in SCM1 human gastric cancer cells. Chin J Physiol 57, 31-40 (2014). https://doi.org: 10.4077/CJP.2014.BAβ141
31 Fang, Y. C. et al. Effect of m-3M3FBS on Ca (2+) movement in Madin-Darby canine renal tubular cells. Hum Exp Toxicol 28, 655-663 (2009). https://doi.org: 10.1177/0960327109106972
32 Krjukova, J., Holmqvist, T., Danis, A. S., Akerman, K. E. & Kukkonen, J. P. Phospholipase C activator m-3M3FBS affects Ca²⁺ homeostasis independently of phospholipase C activation. Br J Pharmacol 143, 3-7 (2004). https://doi.org: 10.1038/sj.bjp.0705911
33 Dwyer, L., Kim, H. J., Koh, B. H. & Koh, S. D. Phospholipase C-independent effects of 3M3FBS in murine colon. Eur J Pharmacol 628, 187-194 (2010). https://doi.org: 10.1016/j.ejphar.2009.11.029
34 Tolvanen, T. A. Current Advances in CETSA. Front Mol Biosci 9, 866764 (2022). https://doi.org: 10.3389/fmolb.2022.866764
35 Scholle, M. D., McLaughlin, D. & Gurard-Levin, Z. A. High-Throughput Affinity Selection Mass Spectrometry Using SAMDI-MS to Identify Small-Molecule Binders of the Human Rhinovirus 3C Protease. SLAS Discov 26, 974-983 (2021). https://doi.org: 10.1177/24725552211023211
36 Huang, W. et al. A High-Throughput Assay to Identify Allosteric Inhibitors of the PLC-gamma Isozymes Operating at Membranes. Biochemistry 59, 4029-4038 (2020). https://doi.org: 10.1021/acs.biochem.0c00511
37 Martinez, N. J. et al. A widely-applicable high-throughput cellular thermal shift assay (CETSA) using split Nano Luciferase. Sci Rep 8, 9472 (2018). https://doi.org: 10.1038/s41598-018-27834-y
38 Oh-Hashi, K., Furuta, E., Fujimura, K. & Hirata, Y. Application of a novel HiBiT peptide tag for monitoring ATF4 protein expression in Neuro2a cells. Biochem Biophys Rep 12, 40-45 (2017). https://doi.org: 10.1016/j.bbrep.2017.08.002
39 Ertl, P., Rohde, B. & Selzer, P. Fast calculation of molecular polar surface area as a sum of fragment-based contributions and its application to the prediction of drug transport properties. J Med Chem 43, 3714-3717 (2000). https://doi.org: 10.1021/jm000942e
40 Lipinski, C. A., Lombardo, F., Dominy, B. W. & Feeney, P. J. Experimental and computational approaches to estimate solubility and permeability in drug discovery and development settings. Adv Drug Deliv Rev 46, 3-26 (2001). https://doi.org: 10.1016/s0169-409x(00) 00129-0
41 Wager, T. T., Hou, X., Verhoest, P. R. & Villalobos, A. Central Nervous System Multiparameter Optimization Desirability: Application in Drug Discovery. ACS Chem Neurosci 7, 767-775 (2016). https://doi.org: 10.1021/acschemneuro.6b00029
42 Bunnage, M. E., Chekler, E. L. P. & Jones, L. H. Target validation using chemical probes. Nature Chemical Biology 9, 195-199 (2013). https://doi.org: 10.1038/nchembio.1197

1. A method of assaying a candidate compound for its tendency to induce phagocytosis in a cell, the method comprising:
(a) contacting the cells with the candidate compound;
(b) contacting the cells with a target ligand bound to a fluorescent phagocytosis vesicle reporter having a fluorophore emission wavelength;
(c) nuclear staining the cells with a DNA-specific fluorescent stain; and
(d) measuring the fluorescence amplitude of the fluorescent phagocytosis vesicle reporter at a measurement wavelength corresponding to the fluorophore emission wavelength.

2. The method of claim 1, wherein the cells are microglia.

3. The method of claim 1, wherein the target ligand is selected from myelin, cell membrane debris, synaptosomes, zymosan, bacteria, protein aggregates, microbeads, and any combination thereof.

4. The method of claim 1, wherein the fluorescent phagocytosis vesicle reporter is a fluorogenic, cell-permeant pH indicator that is non-fluorescent at a neutral pH and increases in intensity with an increase in acidity, and having a peak excitation and emission wavelength at about 560 nm and about 585 nm, respectively.

5. The method of claim 1, wherein the DNA-specific fluorescent stain is Hoechst 33342.

6. The method of claim 1, wherein the candidate compound is analog of 2,4,6-trimethyl-N-(m-tolyl)benzenesulfonamide.

7. A cell-health assay for testing compounds for their potential for treating neurodegenerative disorders, the assay comprising:
(1) plating microglia cells in one or more microplate wells;
(2) incubating the microglia cells;
(3) contacting the microglia cells with one or more candidate compounds;
(4) adding fluorescent-dye-labeled-myelin/membrane debris to the cell plates, wherein the fluorescent-dye-labeled-myelin/membrane debris comprises crude myelin/membrane debris from 6-8-week-old wild type mouse brains isolated via sucrose gradient ultracentrifugation and subsequently labeled with fluorescent dye;
(5) staining the microglia cells' nuclei with a DNA-binding fluorescent dye;
(6) scanning the microglia cells with a fluorescence-reading instrument; and
(7) measuring one or more of (i) mean total fluorescence intensity of phagocytosis vesicles per cell; (ii) cell count per well; and (iii) average nuclear fluorescence intensity.

8. The cell-health assay of claim 7, wherein the microglia cells are BV2 cells, HMC3 cells, a mixture of BV2 cells and HMC3 cells, or mouse primary microglia.

9. The cell-health assay of claim 7, wherein at step (2) the microglia cells are incubated at 37° C. with 5% CO₂ for 16 to 24 hours.

10. The cell-health assay of claim 7, wherein the fluorescent dye is a fluorogenic, cell-permeant dye with an excitation peak at about 560 nm and emission peak at about 585 nm.

11. The cell-health assay of claim 7, wherein step (7) comprises: measuring (i) mean total fluorescence intensity of phagocytosis vesicles per cell; (ii) cell count per well; and (iii) average nuclear fluorescence intensity.

12. The cell-health assay of claim 7, wherein the DNA-binding fluorescent dye is Hoechst 33342.

13. The cell-health assay of claim 7, wherein scanning the microglia cells is done using a high-content analysis (HCA) quantitative cell analysis apparatus having at least a first channel set to scan for an excitation peak of about 386 nm and emission peak of about 460 nm, and a second channel set to scan for an excitation peak of about 560 nm and an emission peak of about 585 nm.

14. A kit for carrying out a phagocytosis assay for testing a compound for its tendency to modulate phagocytosis in microglia, the kit comprising:
one or more well plates;
immortalized microglia cells; and
a ligand for measuring microglial phagocytosis, wherein the ligand is labeled with a fluorogenic, cell-permeant dye with an excitation peak at about 560 nm and emission peak at about 585 nm, which is essentially non-fluorescent or non-fluorescent at neutral pH and which fluoresces at increasing intensity correlated with increasing acidity.

15. The kit of claim 14, wherein the immortalized microglia cells are BV2 cells, HMC3 cells, or a mixture of BV2 cells and HMC3 cells.

16. The kit of claim 14, wherein the ligand is selected from myelin, cell membrane debris, Aβ protein aggregates, Tau protein aggregates, microbeads, zymosan, bacteria, isolated dead neurons, synaptosomes, or any combination thereof.

17. The kit of claim 14, wherein the ligand is myelin/membrane debris isolated from 6-8-week-old wild-type mouse brain.

18. The kit of claim 14, further comprising a cell-membrane-permeable DNA-binding fluorescent dye.

19. The kit of claim 18, wherein the dye is Hoechst 33342.

20. The kit of claim 14, further comprising instructions describing how to perform the assay of claim 7.
